SEQUENCE LISTING (10 sequences)

SEQ ID NO: 1 - protein, 355 aa, Human
MEPHLLGLLLGLLLGGTRVLAGYPIWWSLALGQQYTSLGSQPLLCGSIPGLVPKQLRFCRNYIEIMPSVAEGVKLGIQECQHQFRGRRWNCTTIDDSLAIFGPVLDKATRESAFVHAIASAGVAFAVTRSCAEGTSTICGCDSHHKGPPGEGWKWGGCSEDADFGVLVSREFADARENRPDARSAMNKHNNEAGRTTILDHMHLKCKCHGLSGSCEVKTCWWAQPDFRAIGDFLKDKYDSASEMVVEKHRESRGWVETLRAKYSLFKPPTERDLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 2 - protein, 352 aa, Mouse
MAPLGYLLVLCSLKQALGSYPIWWSLAVGPQYSSLSTQPILCASIPGLVPKQLRFCRNYVEIMPSVAEGVKAGIQECQHQFRGRRWNCTTVSNSLAIFGPVLDKATRESAFVHAIASAGVAFAVTRSCAEGSAAICGCSSRLQGSPGEGWKWGGCSEDIEFGGMVSREFADARENRPDARSAMNRHNNEAGRQAIASHMHLKCKCHGLSGSCEVKTCWWSQPDFRTIGDFLKDKYDSASEMVVEKHRESRGWVETLRPRYTYFKVPTERDLVYYEASPNFCEPNPETGSFGTRDRTCNVSSHGIDGCDLLCCGRGHNARTERRREKCHCVFHWCCYVSCQECTRVYDVHTCK

SEQ ID NO: 3 - protein, 352 aa, Human
MAPLGYFLLLCSLKQALGSYPIWWSLAVGPQYSSLGSQPILCASIPGLVPKQLRFCRNYVEIMPSVAEGIKIGIQECQHQFRGRRWNCTTVHDSLAIFGPVLDKATRESAFVHAIASAGVAFAVTRSCAEGTAAICGCSSRHQGSPGKGWKWGGCSEDIEFGGMVSREFADARENRPDARSAMNRHNNEAGRQAIASHMHLKCKCHGLSGSCEVKTCWWSQPDFRAIGDFLKDKYDSASEMVVEKHRESRGWVETLRPRYTYFKVPTERDLVYYEASPNFCEPNPETGSFGTRDRTCNVSSHGIDGCDLLCCGRGHNARAERRREKCRCVFHWCCYVSCQECTRVYDVHTCK

SEQ ID NO: 4 - protein, 139 aa, Human
MKGESKRITLVLQQPQSGGPQGHRHVVLGSLPGKIVLQGNQLAALTQAKNAQGQPAKVVTIQLQVQQPQQKIQIVPQPPSSQPQPQQPPSTQPVTLSSVQQAQIMGPGQSPGQRLSVPVKVVLQPQRLALPKGPLLGSL

SEQ ID NO: 5 - protein, 433 aa, Human
MSGRPRTTSFAESCKPVQQPSAFGSMKVSRDKDGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKDSSGTGHFTSGVRVFRPRTPPEAIALCSRLLEYTPTARLTPLEACAHSFFDELRDPNVKLPNGRDTPALFNFTTQELSSNPPLATILIPPHARIQAAASTPTNATAASDANTGDRGQTNNAASASASNST

SEQ ID NO: 6 - DNA, 2814 bp, Mouse
gaattcatgtcttacggtcaaggcagagggcccagcgccactgcagccgcgccacctcccagggccgggccagcccaggcgtccgcgctctcggggtggactccccccgctgcgcgctcaagccggcgatggctcctctcggatacctcttagtgctctgcagcctgaagcaggctctgggcagctacccgatctggtggtccttggctgtgggaccccagtactcctctctgagcactcagcccattctctgtgccagcatcccaggcctggtaccgaagcagctgcgcttctgcaggaactacgtggagatcatgcccagcgtggctgagggtgtcaaagcgggcatccaggagtgccagcaccagttccgaggccggcgttggaactgcaccaccgtcagcaacagcctggccatctttggccctgttctggacaaagccacccgggagtcagcctttgtccatgccatcgcctccgctggagtagctttcgcagtgacacgctcctgtgcagagggatcagctgctatctgtgggtgcagcagccgcctccagggctccccaggcgagggctggaagtggggcggctgtagtgaggacattgaatttggaggaatggtctctcgggagtttgccgatgccagggagaaccggccggatgcccgctctgccatgaaccgtcacaacaatgaggctgggcgccaggccatcgccagtcacatgcacctcaagtgcaaatgccacgggctatctggcagctgtgaagtgaagacctgctggtggtcgcagccggacttccgcaccatcggggatttcctcaaggacaagtatgacagtgcctcggagatggtggtagagaaacaccgagagtctcgtggctgggtggagaccctgaggccacgttacacgtacttcaaggtgccgacagaacgcgacctggtctactacgaggcctcacccaacttctgcgaacctaaccccgaaaccggctccttcgggacgcgtgaccgcacctgcaatgtgagctcgcatggcatagatgggtgcgacctgttgtgctgcgggcgcgggcataacgcgcgcactgagcgacggagggagaaatgccactgtgttttccattggtgctgctacgtcagctgccaggagtgcacacgtgtctatgacgtgcacacctgcaagtaggagagctcctaacacgggagcagggttcattccgaggggcaaggttcctacctgggggcggggttcctacttggaggggtctcttacttggggactcggttcttacttgagggcggagatcctacctgtgagggtctcatacctaaggacccggtttctgccttcagcctgggctcctatttgggatctgggttcctttttaggggagaagctcctgtctgggatacgggtttctgcccgagggtggggctccacttggggatggaattccaatttgggccggaagtcctacctcaatggcttggactcctctcttgacccgacagggctcaaatggagacaggtaagctactccctcaactaggtggggttcgtgcggatgggtgggaggggagagattagggtccctcctcccagaggcactgctctatctagatacatgagagggtgcttcagggtgggccctatttgggcttgaggatcccgtgggggcggggcttcaccccgactgggtggaacttttggagacccccttccactggggcaaggcttcactgaagactcatgggatggagctccacggaaggaggagttcctgagcgagcctgggctctgagcaggccatccagctcccatctggcccctttccagtcctggtgtaaggttcaacctgcaagcctcatctgcgcagagcaggatctcctggcagaatgaggcatggagaagaactcaggggtgataccaagacctaacaaaccccgtgcctgggtacctcttttaaagctctgcaccccttcttcaagggctttcctagtctccttggcagagctttcctgaggaagatttgcagtcccccagagttcaagtgaacacccatagaacagaacagactctatcctgagtagagagggttctctaggaatctctatggggactgctaggaaggatcctgggcatgacagcctcgtatgatagcctgcatccgctctgacacttaatactcagatctcccgggaaacccagctcatccggtccgtgatgtccatgccccaaatgcctcagagatgttgcctcactttgagttgtatgaacttcggagacatggggacacagtcaagccgcagagccagggttgtttcaggacccatctgattccccagagcctgctgttgaggcaatggtcaccagatccgttggccaccaccctgtcccgagcttctctagtgtctgtctggcctggaagtgaggtgctacatacagcccatctgccacaagagcttcctgattggtaccactgtgaaccgtccctccccctccagacaggggaggggatgtggccatacaggagtgtgcccggagagcgcggaaagaggaagagaggctgcacacgcgtggtgactgactgtcttctgcctggaactttgcgttcgcgcttgtaactttattttcaatgctgctatatccacccaccactggatttagacaaaagtgattttctttttttttttttcttttctttctatgaaagaaattattttagtttatagtatgtttgtttcaaataatggggaaagtaaaaagagagaaaaaaaaaaaaaaaaaaaaaaaaaaaaaaaaaaaaaaaaaa

SEQ ID NO: 7 - DNA, 1506 bp, Human
gcgcttctgacaagcccgaaagtcatttccaatctcaagtggactttgttccaactattgggggcgtcgctccccctcttcatggtcgcgggcaaacttcctcctcggcgcctcttctaatggagccccacctgctcgggctgctcctcggcctcctgctcggtggcaccagggtcctcgctggctacccaatttggtggtccctggccctgggccagcagtacacatctctgggctcacagcccctgctctgcggctccatcccaggcctggtccccaagcaactgcgcttctgccgcaattacatcgagatcatgcccagcgtggccgagggcgtgaagctgggcatccaggagtgccagcaccagttccggggccgccgctggaactgcaccaccatagatgacagcctggccatctttgggcccgtcctcgacaaagccacccgcgagtcggccttcgttcacgccatcgcctcggccggcgtggccttcgccgtcacccgctcctgcgccgagggcacctccaccatttgcggctgtgactcgcatcataaggggccgcctggcgaaggctggaagtggggcggctgcagcgaggacgctgacttcggcgtgttagtgtccagggagttcgcggatgcgcgcgagaacaggccggacgcgcgctcggccatgaacaagcacaacaacgaggcgggccgcacgactatcctggaccacatgcacctcaaatgcaagtgccacgggctgtcgggcagctgtgaggtgaagacctgctggtgggcgcagcctgacttccgtgccatcggtgacttcctcaaggacaagtatgacagcgcctcggagatggtagtagagaagcaccgtgagtcccgaggctgggtggagaccctccgggccaagtactcgctcttcaagccacccacggagagggacctggtctactacgagaactcccccaacttttgtgagcccaacccagagacgggttcctttggcacaagggaccggacttgcaatgtcacctcccacggcatcgatggctgcgatctgctctgctgtggccggggccacaacacgaggacggagaagcggaaggaaaaatgccactgcatcttccactggtgctgctacgtcagctgccaggagtgtattcgcatctacgacgtgcacacctgcaagtagggcaccagggcgctgggaaggggtgaagtgtgtggctgggcggattcagcgaagtctcatgggaagcaggacctagagccgggcacagccctcagcgtcagacagcaaggaactgtcaccagccgcacgcgtggtaaatgacccagacccaactcgcctgtggacggggaggctctccctctctctcatcttacatttctcaccctactctggatggtgtgtggtttttaaagaagggggctttctttttagttctctagggtctgataggaacagacctgaggcttatctttgcacatgttaaagaaaaaaaaaaaaa

SEQ ID NO: 8 - DNA, 2932 bp, Human
agctcccagggcccggccccccccggcgctcacgctctcggggcggactcccggccctccgcgccctctcgcgcggcgatggccccactcggatacttcttactcctctgcagcctgaagcaggctctgggcagctacccgatctggtggtcgctggctgttgggccacagtattcctccctgggctcgcagcccatcctgtgtgccagcatcccgggcctggtccccaagcagctccgcttctgcaggaactacgtggagatcatgcccagcgtggccgagggcatcaagattggcatccaggagtgccagcaccagttccgcggccgccggtggaactgcaccaccgtccacgacagcctggccatcttcgggcccgtgctggacaaagctaccagggagtcggcctttgtccacgccattgcctcagccggtgtggcctttgcagtgacacgctcatgtgcagaaggcacggccgccatctgtggctgcagcagccgccaccagggctcaccaggcaagggctggaagtggggtggctgtagcgaggacatcgagtttggtgggatggtgtctcgggagttcgccgacgcccgggagaaccggccagatgcccgctcagccatgaaccgccacaacaacgaggctgggcgccaggccatcgccagccacatgcacctcaagtgcaagtgccacgggctgtcgggcagctgcgaggtgaagacatgctggtggtcgcaacccgacttccgcgccatcggtgacttcctcaaggacaagtacgacagcgcctcggagatggtggtggagaagcaccgggagtcccgcggctgggtggagaccctgcggccgcgctacacctacttcaaggtgcccacggagcgcgacctggtctactacgaggcctcgcccaacttctgcgagcccaaccctgagacgggctccttcggcacgcgcgaccgcacctgcaacgtcagctcgcacggcatcgacggctgcgacctgctgtgctgcggccgcggccacaacgcgcgagcggagcggcgccgggagaagtgccgctgcgtgttccactggtgctgctacgtcagctgccaggagtgcacgcgcgtctacgacgtgcacacctgcaagtaggcaccggccgcggctccccctggacggggcgggccctgcctgagggtgggcttttccctgggtggagcaggactcccacctaaacggggcagtactcctccctgggggcgggactcctccctgggggtggggctcctacctgggggcagaactcctacctgaaggcagggctcctccctggagctagtgtctcctctctggtggctgggctgctcctgaatgaggcggagctccaggatggggaggggctctgcgttggcttctccctggggacggggctcccctggacagaggcggggctacagattgggcggggcttctcttgggtgggacagggcttctcctgcgggggcgaggcccctcccagtaagggcgtggctctgggtgggcggggcactaggtaggcttctacctgcaggcggggctcctcctgaaggaggcggggctctaggatggggcacggctctggggtaggctgctccctgagggcggagcgcctccttaggagtggggttttatggtggatgaggcttcttcctggatggggcagagcttctcctgaccagggcaaggccccttccacgggggctgtggctctgggtgggcgtggcctgcataggctccttcctgtgggtggggcttctctgggaccaggctccaatggggcggggcttctctccgcgggtgggactcttccctgggaaccgccctcctgattaaggcgtggcttctgcaggaatcccggctccagagcaggaaattcagcccaccagccacctcatccccaaccccctgtaaggttccatccacccctgcgtcgagctgggaaggttccatgaagcgagtcgggtccccaacccgtgcccctgggatccgagggcccctctccaagcgcctggctttggaatgctccaggcgcgccgacgcctgtgccaccccttcctcagcctggggtttgaccacccacctgaccaggggccctacctggggaaagcctgaagggcctcccagcccccaaccccaagaccaagcttagtcctgggagaggacagggacttcgcagaggcaagcgaccgaggccctcccaaagaggcccgccctgcccgggctcccacaccgtcaggtactcctgccagggaactggcctgctgcgccccaggccccgcccgtctctgctctgctcagctgcgcccccttctttgcagctgcccagcccctcctccctgccctcgggtctccccacctgcactccatccagctacaggagagatagaagcctctcgtcccgtccctccctttcctccgcctgtccacagccccttaagggaaaggtaggaagagaggtccagccccccaggctgcccagagctgctggtctcatttgggggcgttcgggaggtttggggggcatcaaccccccgactgtgctgctcgcgaaggtcccacagccctgagatgggccggcccccttcctggcccctcatggcgggactggagaaatggtccgctttcctggagccaatggcccggcccctcctgactcatccgcctggcccgggaatgaatggggaggccgctgaacccacccggcccatatccctggttgcctcatggccagcgcccctcagcctctgccactgtgaaccggctcccaccctcaaggtgcggggagaagaagcggccaggcggggcgccccaagagcccaaaagagggcacaccgccatcctctgcctcaaattctgcgtttttggttttaatgttatatctgatgctgctatatccactgtccaacgg

SEQ ID NO: 9 - DNA, 923 bp, Human
gagttccaccatcttcctctgaagacgtagccatcttgctccatgaagggtgaatcgaaacgcatcaccctggtcctccagcagccacagtctggaggtccccaaggacatcggcatgttgtgctagggagtctaccaggcaagatagtgttacagggcaaccagctggcagccctgactcaagccaagaatgcccaagggcagcctgccaaggtagtaactatccagctgcaggtgcagcagccacagcaaaaaatccagattgtaccacaaccaccatcatcgcagccacagccccagcagccaccctccacccagccagtgactctgtcctctgtacagcaggctcagataatgggaccaggacaaagcccaggacaaagactttcagtaccagtcaaggtggtactgcagccacagaggctggctcttcccaaggggcctcttctgggctctctgtagttaaagttctgagtgccagtgaagtggcagctttgtcatcaccagcaagctctgctcctcattcggggggaaagacaggaatggaggaaaaccgcagattggaacaccagaagaagcaagagaaagcaaatcggattgtagcagaggccattgcgagagcccgtgcccgcggtgagcagaacatacctcgagtcttaaatgaggacgagttgcccagcgttcggccagaggaggaaggcgagaagaaacgcaggaagaagagtgctggggagaggctgaaagaggagaagccaaagaagagtaaaacatctggtgcctccaaaacaaagggcaagagcaagctcaacaccatcactcctgtagtgggtaagaagagaaaacgtaatacctcatctgataattcagatgtggaagtcatgcctgcacagtcacctcgagaagatgaagaaagcagcattcag

SEQ ID NO: 10 - DNA, 1639 bp, Human
atcatctatatgttaaatatccgtgccgatctgtcttgaaggagaaatatatcgcttgttttgttttttatagtatacaaaaggagtgaaaagccaagaggacgaagtctttttctttttcttctgtgggagaacttaatgctgcatttatcgttaacctaacaccccaacataaagacaaaaggaagaaaaggaggaaggaaggaaaaggtgattcgcgaagagagtgatcatgtcagggcggcccagaaccacctcctttgcggagagctgcaagccggtgcagcagccttcagcttttggcagcatgaaagttagcagagacaaggacggcagcaaggtgacaacagtggtggcaactcctgggcagggtccagacaggccacaagaagtcagctatacagacactaaagtgattggaaatggatcatttggtgtggtatatcaagccaaactttgtgattcaggagaactggtcgccatcaagaaagtattgcaggacaagagatttaagaatcgagagctccagatcatgagaaagctagatcactgtaacatagtccgattgcgttatttcttctactccagtggtgagaagaaagatgaggtctatcttaatctggtgctggactatgttccggaaacagtatacagagttgccagacactatagtcgagccaaacagacgctccctgtgatttatgtcaagttgtatatgtatcagctgttccgaagtttagcctatatccattcctttggaatctgccatcgggatattaaaccgcagaacctcttgttggatcctgatactgctgtattaaaactctgtgactttggaagtgcaaagcagctggtccgaggagaacccaatgtttcgtatatctgttctcggtactatagggcaccagagttgatctttggagccactgattatacctctagtatagatgtatggtctgctggctgtgtgttggctgagctgttactaggacaaccaatatttccaggggatagtggtgtggatcagttggtagaaataatcaaggtcctgggaactccaacaagggagcaaatcagagaaatgaacccaaactacacagaatttaaattccctcaaattaaggcacatccttggactaaggattcgtcaggaacaggacatttcacctcaggagtgcgggtcttccgaccccgaactccaccggaggcaattgcactgtgtagccgtctgctggagtatacaccaactgcccgactaacaccactggaagcttgtgcacattcattttttgatgaattacgggacccaaatgtcaaactaccaaatgggcgagacacacctgcactcttcaacttcaccactcaagaactgtcaagtaatccacctctggctaccatccttattcctcctcatgctcggattcaagcagctgcttcaacccccacaaatgccacagcagcgtcagatgctaatactggagaccgtggacagaccaataatgctgcttctgcatcagcttccaactccacctgaacagtcccgagcagccagctgcacaggaaaaaccaccagttacttgagtgtcactcagcaacactggtcacgtttggaaagaatattaaaaaaaaaaaaaaaaaa

(19) United States
(12) Patent Application Publication
(10) Pub. No.: US 2005/0043260 A1
(43) Pub. Date: Feb. 24, 2005

(54) Wnt as a factor for cardiac myogenesis

(75) Inventors: Michael D. Schneider, Houston, TX (US); Teruya Nakamura, Houston, TX (US)

Correspondence Address: FULBRIGHT & JAWORSKI, LLP, 1301 MCKINNEY, SUITE 5100, HOUSTON, TX, 77010-3095, US

(73) Assignee: BAYLOR COLLEGE OF MEDICINE, Houston, TX (US)

(21) Appl. No.: 10/828,669

(22) Filed: Apr. 21, 2004

Related U.S. Application Data
(60) Provisional application No. 60/464,292, filed on Apr. 21, 2003.

(51) Int. Cl. A61K 48/00; C12N 15/85

(52) U.S. Cl. 514/44; 435/455

(57) ABSTRACT

The present invention relates to the fields of cell biology, molecular biology, and medicine. More specifically, the invention is directed to generating cardiomyocyte cells from non-cardiomyocyte cells by enhancing the activation of the Wnt/β-catenin signaling pathway. The cardiomyocyte cells that are generated in the present invention are then used as cardiac disease therapy.

### CROSS-REFERENCE TO RELATED APPLICATIONS

This application claims priority to U.S. Provisional Application No. 60/464,292 filed Apr. 21, 2003, which is incorporated by reference herein in its entirety.

### STATEMENT REGARDING FEDERALLY SPONSORED RESEARCH OR DEVELOPMENT

This invention was made with government support under NHLBI Grant No. HL49953 awarded by the National Institutes of Health. The United States Government may have certain rights in the invention.

### TECHNICAL FIELD

The present invention relates to the fields of cell biology, molecular biology, and medicine. More specifically, the invention is directed to generating cardiomyocyte cells from non-cardiomyocyte cells by enhancing the activation of the Wnt/β-catenin signaling pathway. The cardiomyocyte cells that are generated in the present invention are then used as cardiac disease therapy.

### BACKGROUND OF THE INVENTION

Cardiovascular disease involves diseases or disorders associated with the cardiovascular system. Such disease and disorders include those of the pericardium, heart valves, myocardium, blood vessels, and veins.

Cell transplantation has emerged as a potential novel approach for regeneration of damaged myocardium. Transplantation of xenogeneic, allogeneic, and autologous cardiomyocytes, skeletal muscle cells, and smooth muscle cells in normal and injured myocardium has been reported in different species. Several studies have demonstrated the feasibility of engrafting exogenous cells into host myocardium, including fetal cardiomyocytes (Soonpaa et al., 1994), cardiomyocytes derived from artial tumor (ATI) (Koh et al., 1993), satellite cells (Chiu et al., 1995), or bone marrow cells (Tomita et al., 1999). These engrafted cells have been histologically identified in normal myocardium up to 4 months after transplantation (Koh et al., 1993). Cells transplanted close to native cardiomyocytes could form intercalated disks. Gap junctions have been found between the engrafted fetal cardiomyocytes and the host myocardium (Soonpaa et al., 1994), thereby raising the possibility of an electrical contraction coupling between transplanted cells and the host tissue. Recently, cell transplantation has been extended into ischemically damaged myocardium in rats with coronary artery occlusion (Scorsin et al., 1996; 2000), or in cryoinjured rats (Li et al., 1996) and dogs (Chiu et al., 1995). More recently, Li and his coworkers (Li et al., 2000) showed that autologous porcine heart cell transplantation improved regional perfusion and global ventricular function after a myocardial infarction.

Over the last two decades, the morbidity and mortality of heart failure has markedly increased (Tavazzi, 1998). Therefore, finding an effective therapeutic method is one of the greatest challenges in public health for this century. Although there are several alternative ways for treatment of heart failure, such as coronary artery bypass grafting and whole-heart transplantation, myocardial fibrosis and organ shortage, along with strict eligibility criteria, mandate the search for new approaches to treat the disease. Cell transplantation has emerged to be able to increase the number of contractile myocytes in damaged hearts.

Thus, it is necessary to develop alternatives to the presently used transplantation techniques. In light of this need, the present invention is the first to use non-cardiomyocyte cells that have been differentiated and/or converted into cardiomyocytes, which can then be used to treat damaged cardiovascular tissue.

### BRIEF SUMMARY OF THE INVENTION

The present invention is directed to a system, method, and compositions related to cardiomyocyte differentiation from a non-cardiomyocyte cell, also referred to herein as a progenitor cell, using factors that activate Wnt/β-catenin signaling.

An embodiment of the present invention is a method of upregulating expression of a cardiac specific polynucleotide in a cell, comprising the step of delivering a composition that activates Wnt/β-catenin signaling. The cell can be in a tissue, specifically, the tissue can be in a mammal, more specifically, the mammal can be a human. After the composition is delivered to the cell, it is envisioned that the cell exhibits spontaneous cell beating.

In certain aspects, the cardiac specific polynucleotide is selected from the group consisting of Nkx2.5, GATA4, MEF2C, Tbx5, CRIPTO, NODAL, and cardiac myosin heavy chain.

Still further, the composition can be a modulator of Wnt. More specifically, the modulator enhances expression of Wnt and/or the modulator enhances activity of Wnt. It is envisioned that the composition delivers Wnt to the cell. For example, Wnt can be delivered to the cell as a polynucleotide and/or a polypeptide. The polynucleotide can be comprised in a vector, (i.e., a viral vector and/or non-viral vector). More specifically, the viral vector is an adenoviral vector, an adeno-associated vector, a retroviral vector or a lentiviral vector.

In further aspects, the composition is a modulator of β-catenin. The modulator enhances accumulation of β-catenin and/or inhibits phosphorylation of β-catenin. Yet further, the composition is an inhibitor of glycogen synthase kinase 3β. More specifically, the composition is lithium.

Another embodiment of the present invention comprises a method of upregulating expression of a cardiac specific polynucleotide in a cell, comprising the step of delivering Wnt to the cell.

Still further, another embodiment is a method of enhancing proliferation or differentiation of a cardiomyocyte cell from a non-cardiomyocyte cell, comprising the step of delivering a composition to the non-cardiomyocyte cell that activates Wnt/β-catenin signaling. The non-cardiomyocyte cell can be derived from autologous tissue, allogeneic tissue, and/or xenogeneic tissue.

It is envisioned that the non-cardiomyocyte cell can be a fibroblast, a stem cell, a progenitor cell. More specifically, the non-cardiomyocyte can be obtained from bone marrow, umbilical cord blood, umbilical tissue, circulating endothelial progenitor cells, cardiac fibroblasts, adipose tissue or skin.

In certain embodiments, the cardiomyocyte cell is defined as a cell comprising at least one of the following: expression of Nkx2.5; expression of GATA4; expression of Tbx5; expression of MEF2C; and expression of cardiac myosin heavy chain.

Another embodiment is a method of treating cardiovascular disease (i.e., heart failure) in a subject, said subject comprising a cell, comprising the step of delivering a composition that activates Wnt/β-catenin signaling to the cell. The method is further defined as: obtaining a cell from the subject; delivering the composition to activate Wnt/β-catenin signaling to the cell; growing the cell to form a cell culture; and delivering at least one cell from the cell culture to said individual. The step of delivering at least one cell from the cell culture to the subject is further defined as: generating a tissue from said at least one cell from said cell culture; and administering said tissue to said subject.

Another embodiment is a method of generating myocytes comprising the steps of: obtaining non-cardiomyocyte cells; admixing a composition that activates Wnt/β-catenin signaling; and in vitro differentiating the cells to generate myocytes. The step obtaining the non-cardiomyocyte cells comprises performing a tissue biopsy. The tissue is bone marrow, umbilical cord blood, umbilical tissue, circulating endothelial progenitor cells, cardiac fibroblasts, adipose tissue or skin.

Still further, another method of treating a subject suffering from an infarcted myocardium comprising the step of administering to the subject an effective amount of the myocytes of present invention, wherein the amount repairs the infarcted myocardium. The repairs comprise regeneration of cardiomyocytes.

Another aspect is a method of repairing an injured myocardium comprising the step of administering to a subject an effective amount of the myocytes of present invention, wherein the amount is effective in repairing the injured myocardium. The step of repairing comprises at least partially restoring structural integrity to the injured myocardium and/or at least partially restoring functional integrity to the injured myocardium.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

The patent or application file contains at least one drawing executed in color. Copies of this patent or patent application publication with color drawing(s) will be provided by the Office upon request and payment of the necessary fee.

FIG. 1A-1D show Wnt3A and Wnt8 are early responses in differentiating P19CL6 cells. FIG. 1A shows gene expression as determined by QRT-PCR, normalized to GAPDH, and represented as relative copy number. FIG. 1B is a Western Blot showing induction of GATA4 and sarcomeric MHC, versus tubulin as the control. FIG. 1C uses immunocytochemistry to show induction of sarcomeric MHC. Bar, 50 μm. FIG. 1D shows FGF8 expression by RT-PCR.

FIG. 2A-2D show that the Wnt/β-catenin-signaling pathway is activated at the onset of cardiac myogenesis. FIG. 2A shows an increased soluble β-catenin. Cells were treated with (+) or without (−) 1% DMSO for 3 days. The cytosolic fraction was subjected to Western blot analysis. β-catenin was specifically decreased by Fz-8/Fc, an extracellular antagonist of Wnt signaling. FIG. 2B shows a decreased phosphorylated β-catenin and increased total β-catenin in whole cell lysates. Cells were cultured as above and Western blotting was done using antibodies to phospho-β-catenin (top) and total β-catenin (middle). FIG. 2C shows decreased phosphorylated β-catenin, shown by immunostaining. Left, without DMSO; right, with DMSO. Nuclei were counterstained with DAPI. Bar; 50 μm. FIG. 2D shows TCF/LEF-dependent transcription. Cells were cultured±DMSO, and transfected with TOPFLASH or FOPFLASH (inactive, mutant TCF sites) along with pRLCMV. Luciferase activity was determined after 4 days of treatment.

FIG. 3A-3F show that the Wnt/β-catenin signaling pathway was required for cardiac differentiation. FIG. 3A shows that Fz-8/Fc suppresses the induction of GATA4, TBX5, BMP2, BMP4, FGF8, α-MHC as determined by QRT-PCR. Cells were treated with DMSO plus diluent or 200 ng/ml Fz-8/Fc. Equivalent results were obtained using Fz-4/Fc. FIG. 3B shows that Fz-8/Fc suppresses the induction of sarcomeric MHC protein. FIG. 3C shows constitutively active GSK-3β (GSK3βA9) suppresses the induction of sarcomeric MHC. Bar, 25 μm. FIG. 3D shows that GSK3βA9 inhibits Wnt3A-induced TCF transcriptional activity. 293T cells were co-transfected with PGK-neo, PGK-Wnt3A, and pcDNA3-GSK3βA9-HA as shown, plus the TOPFLASH and pRL-CMV reporter genes. FIG. 3E shows GSK3βA9-HA expression in stably-transformed P19CL cells as determined by RT-PCR. FIG. 3F shows that GSK3βA9 suppresses DMSO-induced cardiac gene expression.

FIG. 4A-4D show that the Wnt/β-catenin signaling pathway enhances cardiac myogenesis. FIG. 4A shows that Wnt3A CM increases TCF-dependent transcription. P19CL6 cells co-transfected with TOPFLASH and pRL-CMV were assayed after 18 hr in Wnt3A or control (neo) CM. FIG. 4B shows that Wnt3A CM induces cardiac-specific markers (0-15 day) and BMPs (6 day) as shown by QRT-PCR. FIG. 4C shows that Wnt3A CM induces sarcomeric MHC as shown by immunostaining. Bar; 50 μm. FIG. 4D shows that LiCl enhances cardiomyogenesis. Induction of cardiac-specific markers was analyzed by QRT-PCR (day 5). NaCl was added as the control.

FIG. 5 shows a list of Wnt-dependent early genes and Wnt-inhibited early genes that were identified in P19C16 cells.

### DETAILED DESCRIPTION OF THE INVENTION

### I. Definitions

The term “a” or “an” as used herein in the specification may mean one or more. As used herein in the claim(s), when used in conjunction with the word “comprising”, the words “a” or “an” may mean one or more than one, but it is also consistent with the meaning of “one or more”, “at least one”, and “one or more than one” Still further, the terms “having”, “including”, “containing”, and “comprising”, are interchangeable and one of skill in the art is cognizant that these terms are open ended terms.

As used herein “another” may mean at least a second or more.

As used herein, the term “allogeneic” refers to tissue or cells derived from another subject of the same species.

As used herein, the term “autologous” refers to tissue or cells that are derived from the same subject's body.

As used herein, the term “blood vessel” as used herein is defined as a vessel or canal through which blood circulates. Examples include artery, vein, and capillary.

As used herein, the term “cardiovascular disease” is defined as a medical condition related to the cardiovascular (heart) or circulatory system (blood vessels). Cardiovascular disease includes, but is not limited to, diseases and/or disorders of the pericardium, heart valves (i.e., incompetent valves, stenosed valves, rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). Yet further, one skilled in the art recognizes that cardiovascular diseases can result from congenital defects, genetic defects, environmental influences (i.e., dietary influences, lifestyle, injury, stress, etc.), and other defects or influences, and combinations thereof.

As used herein, the term “cardiovascular tissue” is defined as heart tissue and/or blood vessel tissue.

As used herein, the term “cardiomyocyte” or “cardiac cell” is meant any cell in the cardiac myocyte lineage that shows at least one phenotypic characteristic of a cardiac muscle cell. Such phenotypic characteristics can include expression of cardiac proteins, such as cardiac sarcomeric or myofibrillar proteins or atrial natriuretic factor. Other characteristics can include electrophysiological characteristics

As used herein, the term “coronary artery disease” (CAD) refers to a type of cardiovascular disease. CAD is caused by gradual blockage of the coronary arteries. One of skill in the art realizes that in coronary artery disease, atherosclerosis (commonly referred to as “hardening of the arteries”) causes thick patches of fatty tissue to form on the inside of the walls of the coronary arteries. These patches are called plaques. As a plaque thickens, the artery narrows and blood flow decreases, which results in a decrease in oxygen to the myocardium. This decrease in blood flow precipitates a series of consequences for the myocardium. For example, interruption in blood flow to the myocardium results in an “infarct” (myocardial infarction), which is commonly known as a heart attack.

As used herein, the term “damaged myocardium” refers to myocardial cells that have been exposed to ischemic conditions. These ischemic conditions may be caused by a myocardial infarction, or other cardiovascular disease or related complaint. The lack of oxygen causes the death of the cells in the surrounding area, leaving an infarct, which eventually scars.

As used herein, the term “DNA” is defined as deoxyribonucleic acid.

As used herein, the term “expression construct” or “transgene” is defined as any type of genetic construct containing a nucleic acid coding for gene products in which part or all of the nucleic acid encoding sequence is capable of being transcribed can be inserted into the vector. The transcript is translated into a protein, but it need not be. In certain embodiments, expression includes both transcription of a gene and translation of mRNA into a gene product. In other embodiments, expression only includes transcription of the nucleic acid encoding genes of interest. In the present invention, the term “therapeutic construct” may also be used to refer to the expression construct or transgene. One skilled in the art realizes that the present invention utilizes the expression construct or transgene as a therapy to treat heart disease, thus the expression construct or transgene is a therapeutic construct.

As used herein, the term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well and are described infra.

The term “fibroblast” as used herein is defined as a connective tissue cell, which is usually a flat elongated cell with cytoplasmic processes at each end, having a flat, oval, vesicular nucleus. A skilled artisan recognizes that fibroblasts, which differentiate endogenously into chondroblasts, collagenoblasts, and osteoblasts, form the fibrous tissues in the body, tendons, aponeuroses, supporting and binding tissues of all sorts. In a specific embodiment, a fibroblast is also referred to as a fibrocyte or a desmocyte.

As used herein, the term “gene” is defined as a functional protein, polypeptide, or peptide-encoding unit. As will be understood by those in the art, this functional term includes genomic sequences, cDNA sequences, and smaller engineered gene segments that express, or is adapted to express, proteins, polypeptides, domains, peptides, fusion proteins, and mutants.

As used herein, the term “infarct” or “myocardial infarction (MI)” refers to an interruption in blood flow to the myocardium. Thus, one of skill in the art refers to MI as death of cardiac muscle cells resulting from inadequate blood supply.

As used herein, the term “ischemic heart disease” refers to a lack of oxygen due to inadequate perfusion or blood supply. Ischemic heart disease is a condition having diverse etiologies. One specific etiology of ischemic heart disease is the consequence of atherosclerosis of the coronary arteries.

As used herein, the term “modulator” refers to a compound that activates the Wnt/β-catenin signaling pathway. For example, the modulator increases or enhances Wnt and/or β-catenin activity or inhibits or blunts GSK-3β activity. The modulator of Wnt and/or β-catenin may also be referred to as an “activator” or “effector” of Wnt and/or β-catenin that can effect or regulate activity of Wnt and/or β-catenin or expression of Wnt and/or β-catenin at any point along the pathway, for example, but not limited to increasing the expression and/or activity of Wnt dependent genes and/or proteins, increases in cardiac myogenesis, etc. The modulator of GSK-3β may also be referred to as an “inhibitor” that can inhibit activity GSK-3β and/or expression of GSK-3β at any point along the pathway, for example, but not limited to prohibiting phosphorylation of β-catenin leading to an accumulation of β-catenin and an association of β-catenin with TCF/LEF and/or an increase in the expression and/or activity of Wnt dependent genes, and/or an increase in cardiac myogenesis. Thus, one of skill in the art recognizes that the modulators of the present invention maintain or regulate Wnt signal transduction at any point along the known Wnt/β-catenin pathway, or yet undiscovered pathway, including but not limiting to cardiac myogenesis, association of proteins with transcription factors and/or cardiac specific genes, increasing expression and/or activity of enzymes, increasing expression and/or activity of Wnt dependent genes or proteins, decreasing expression and/or activity of known inhibitors or yet undiscovered inhibitors of Wnt and/or β-catenin and/or Wnt dependent genes, increasing expression and/or activity of known activators or yet undiscovered activators of Wnt and/or β-catenin and/or Wnt dependent genes, etc.

As used herein, the term “myocardium” refers to the muscle of the heart.

As used herein, the term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

As used herein, the term “palliating” a disease means that the extent or undesirable clinical manifestations of a disease state are lessened and/or the time course of the progression is slowed or lengthened, as compared to the disease in the absence of the substance and/or composition of the present invention.

As used herein, the term “polynucleotide” is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means. Furthermore, one skilled in the art is cognizant that polynucleotides include mutations of the polynucleotides, include but are not limited to, mutation of the nucleotides, or nucleosides by methods well known in the art.

As used herein, the term “polypeptide” is defined as a chain of amino acid residues, usually having a defined sequence. As used herein the term polypeptide is interchangeable with the terms “peptides” and “proteins”.

As used herein, the term “promoter” is defined as a DNA sequence recognized by the synthetic machinery of the cell, or introduced synthetic machinery, required to initiate the specific transcription of a gene.

As use herein, the term “progenitor cell” refers to a cell that is an undifferentiated cell that is capable of differentiating. One of skill in the art realizes that a progenitor cell is an ancestor cell to progeny descendant cells.

As used herein, the term “stem cells” refers to “undifferentiated” cells capable of proliferation, self-maintenance, production of differentiated cells and/or regeneration of stem cells. In preferred embodiments of the present invention, a stem cell is capable of differentiating into a differentiated cell, such as a cardiomyocyte.

As used herein, the term “subject” may encompass any vertebrate including but not limited to mammals, reptiles, amphibians and fish. However, advantageously, the subject is a mammal such as a human, or other mammals such as a domesticated mammal, e.g., dog, cat, horse, and the like, or production mammal, e.g., cow, sheep, pig, and the like.

As used herein, the term “therapeutically effective amount” refers to an amount that results in an improvement or remediation of the disease, disorder, or symptoms of the disease or condition.

As used herein, the term “treating” and “treatment” and/or “palliating” refers to administering to a subject an effective amount of a the composition so that the subject has an improvement in the disease, for example, beneficial or desired clinical results. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Thus, one of skill in the art realizes that a treatment may improve the disease condition, but may not be a complete cure for the disease. As used herein, the term “treatment” includes prophylaxis.

As used herein, the term “RNA” is defined as ribonucleic acid.

As used herein, the term “under transcriptional control” or “operatively linked” is defined as the promoter is in the correct location and orientation in relation to the nucleic acid to control RNA polymerase initiation and expression of the gene.

As used herein, the term “xenogeneic” refers to cells that are derived from different species.

### II. Cell Proliferation or Differentiation

The present invention is directed to cell differentiation of a particular cell type, such as a non-cardiomyocyte cell into a cardiac cell or cardiomyocyte cell and, in a preferred embodiment, the cardiomyocyte and/or a tissue comprising the same is used for cardiac disease therapy.

Enhancing proliferation of a cell encompasses the step of increasing the extent of growth and/or reproduction of the cell relative to an untreated cell either in vitro or in vivo. An increase in cell proliferation in cell culture can be detected by counting the number of cells before and after exposure to a molecule of interest. The extent of proliferation can be quantified via microscopic examination of the degree of confluency. Cell proliferation can also be quantified using a thymidine incorporation assay.

Enhancing differentiation of a cell is meant as the act of increasing the extent of the acquisition or possession of one or more characteristics or functions which differ from that of the original cell (i.e., cell specialization). This can be detected by screening for a change in the phenotype of the cell (i.e., identifying morphological changes in the cell and/or surface markers on the cell).

Enhancing conversion of a non-cardiomyocyte cell into a cardiomyocyte cell as used in the present invention is meant to include the act of increasing the extent of the acquisition or possession of one or more characteristics or functions which are used to identify a cell as a cardiomyocyte. For example, a specific function can include spontaneous beating, however, the present invention is not limited to this function of spontaneous beating. As used in the present invention, the term differentiation and conversion may be interchangeable.

The non-cardiomyocyte cells used in the present invention include non-cardiomyocyte cells that are capable of differentiating into a cardiomyocyte cell or has “cardiomyocyte potential”. By “cardiomyocyte potential” is meant to refer to the ability to give rise to progeny that can differentiate into a cardiomyocyte under specific conditions. Examples of non-cardiomyocyte cells with cardiomyocyte potential include pluripotent cells, progenitor cells (i.e., circulating endothelial progenitor cells or hemangioblasts), stem cells (i.e., hematopoietic stem cells, embryonic stem cells, or Sca1 cells (See U.S. patent application Ser. No. 10/651,548 filed Aug. 29, 2003, which is incorporated by reference herein in its entirety), or fibroblast (i.e., muscle fibroblast, cardiac fibroblast, etc.). These non-cardiomyocyte cells can be isolated from embryonic or nonembryonic donors from, e.g., the bone marrow, the spleen, the liver, peripheral blood, umbilical cord tissue, umbilical cord blood, adipose tissue or skin. The donor tissue or sample can be isolated from a vertebrate, more particularly a mammal, for example human, dog, cat, monkey, mouse, rat, bird, etc. More preferably the mammal is an adult mammal. In preferred embodiments, the mammal is a human. The tissue and/or sample can include the entire tissue or sample, a portion of a tissue or sample, or biopsy sample.

Any method of isolating non-cardiomyocyte cells is acceptable, including affinity-based interactions, affinity panning, or flow cytometry. Cell density gradients (e.g., Ficoll-Paque), cell sorting using cell surface antigens (e.g., with a fluorescence activated cell sorter), or by magnetic beads (StemSep Technologies) which bind cells with certain cell surface antigens can be used to separate mature cells from non-mature cells. These methods can be combined to yield a relatively pure population of desired mature non-cardiomyocyte cells for use in the present invention. For example, the Ficoll Paque method and the magnetic bead method can be combined to yield single non-cardiomyocyte cell population with a purity of greater than about 85% and optimally of about 99% purity.

By “relatively pure population” is meant a population of cells comprising at least about 80% cells with cardiomyocyte cell potential. More preferably, the population comprises at least about 90% cells with cardiomyocyte cell potential. Even more preferably, the population comprises at least about 95% cells with cardiomyocyte cell potential. Most preferably, the population comprises at least about 99% cells with cardiomyocyte cell potential.

### III. Modulators of Wnt/β-Catenin Signaling Pathway

Wnt/Wg genes, related to wingless in Drosophila, encode a number of secreted proteins that play critical roles in development of many organisms, especially in cell fate and patterning (Arias et al., 1999; Bejsovec, 1999; Moon et al., 2002). Notably, the prototype, wingless itself, collaborates with the BMP homologue, decapentaplegic, to specify the rudimentary heart tube in flies (Wu et al., 1995; Park et al., 1995; Frasch et al., 1995; Lee et al.,2000). In absence of Wnt proteins, cells undertake active measures to maintain low levels of the Wnt signaling protein, β-catenin.

β-catenin is a pivotal player in the signaling pathway initiated by Wnt proteins, which are mediators of several developmental processes. β-catenin activity is controlled by a large number of binding partners that affect the stability and the localization of β-catenin, and it is thereby able to participate in such varying processes as gene expression and cell adhesion. Activating mutations in β-catenin and in components regulating its stability have been found to contribute to upregulation of cell proliferation.

The β-catenin protein becomes stabilized in response to Wnt/Wg, moves to the nucleus and forms complexes with the LEF1/TCF transcription factors to regulate gene expression. The level of cytosolic β-catenin is determined by its interaction with a number of proteins including those in a multiprotein complex of Axin, GSK-3β, APC and other proteins. The mechanism by which the Wnt signal is transmitted to this complex is unclear but it involves interaction of Wnt with its receptors, which are members of Frizzled family of seven transmembrane proteins. The stabilization of β-catenin stimulates the expression of genes including c-myc, c-jun, fra-1, and cyclin D1. This pathway is negatively regulated by Axin.

The ubiquitin-dependent proteolysis system is involved in the regulation of beta-catenin turnover. Beta-catenin becomes stabilized when proteasome-mediated proteolysis is inhibited and this leads to the accumulation of multi-ubiquitinated forms of beta-catenin (Aberle et al., 1997). Substitution of the serine residues in the glycogen synthase kinase 3β. (GSK-3β) phosphorylation consensus motif of β-catenin inhibits ubiquitination and results in stabilization of the protein. Examples of stabilized β-catenins include those with the amino acid changes D32Y; D32G; S33F; S33Y; G34E; S37C; S37F; T41I; S45Y; and deletion of AA 1-173. A number of publications describe stabilized β-catenin mutations. (For example, see Morin et al., 1997; Palacios et al., 1998; Muller et al., 1998; Miyoshi et al., 1998; Zurawel et al., 1998; Voeller et al., 1998; and U.S. Pat. No., 6,465,249, etc., which are incorporated herein by reference in their entirety.

A. Modulators

In certain embodiments, modulators of the Wnt/β-catenin signaling are administered to a subject to enhance the activity and/or expression of Wnt and/or β-catenin. Yet further modulators of GSK-3β are administered to a subject to suppress the activity and/or expression of GSK-3β resulting in an accumulation of β-catenin and an increased or enhanced activity of the Wnt/β-catenin signaling pathway.

The modulators of the present invention include, but are not limited to polynucleotides, polypeptides, antibodies, small molecules or other compositions that are capable of modulating either the activity and/or the expression of Wnt, Wnt-dependent genes/proteins, Wnt-inhibited genes/proteins, β-catenin and/or GSK-3β.

In the present invention, the terms “Wnt gene product” or “β-catenin gene product” or “GSK-3β gene product” refer to proteins and polypeptides having amino acid sequences that are substantially identical to the native Wnt, β-catenin, or GSK-3β amino acid sequences (or RNA, if applicable) or that are biologically active, in that they are capable of performing functional activities similar to an endogenous Wnt, β-catenin, or GSK-3β, and/or cross-reacting with anti-Wnt antibody raised against Wnt, and/or cross-reacting with anti-β-catenin antibody raised against β-catenin, and/or cross-reacting anti-GSK-3β antibody raised against GSK-3β.

The terms “Wnt gene product” or “β-catenin gene product” or “GSK-3β gene product” also include analogs of the respective molecules that exhibit at least some biological activity in common with their native counterparts. Such analogs include, but are not limited to, truncated polypeptides and polypeptides having fewer amino acids than the native polypeptide.

In specific embodiments, the term “Wnt polypeptide” includes human Wnt polypeptides, Wnt-1, 2A, 2B, 3, 3A, 4, 5A, 5B, 7A, 7B, 8A, 8B, 9A, 9B, 10A, 10B, 11A, and murine Wnt polypeptides, Wnt-1, 2, 3A, 3B, 4, 5A, 5B, 6, 7A, 7B, 8A, 8B, 10B, 11 and 12. (See U.S. Pat. Nos. 5,851,984 and 6,159,462, which are incorporated herein by reference in their entirety). More preferably, the Wnt polypeptide sequences include sequences related to Wnt3A, for example, but not limited to SEQ.ID.NO.1 (GenBank accession #NP_{—}110380), SEQ.ID.NO.2 (GenBank accession #NP_{—}033548); and/or SEQ.ID.NO.NO.3 (GenBank accesion #NP_{—}149122).

β-catenin polypeptide sequences include, but are not limited to SEQ.ID.NO.4 (GenBank accession #XP_{—}208760). The GSK-3β polypeptide sequences include, but are not limited to, SEQ.ID.NO.5 (GenBank accession #NP_{—}002084).

The term “Wnt gene” “Wnt polynucleotide” or “Wnt nucleic acid” refers to any DNA sequence that is substantially identical to a DNA sequence encoding a Wnt gene product as defined above. Similar terms for β-catenin or GSK-3β are within the scope of the present invention. The term also refers to RNA or antisense sequences compatible with such DNA sequences. As used herein the term gene or polynucleotide may also comprise any combination of associated control sequences.

In specific embodiments, the term “Wnt polynucleotide” includes human Wnt polynucleotides, Wnt-1, 2A, 2B, 3, 3A, 4, 5A, 5B, 7A, 7B, 8A, 8B, 9A, 9B, 10A, 10B, 11A, and murine Wnt polynucleotides, Wnt-1, 2, 3A, 3B, 4, 5A, 5B, 6, 7A, 7B, 8A, 8B, 10B, 11 and 12. (See U.S. Pat. Nos. 5,851,984 and 6,159,462, which are incorporated herein by reference in their entirety). More preferably, Wnt polynucleotide sequences include sequences related to Wnt3A, for example, but not limited to SEQ.ID.NO.6 (GenBank accession #NM_{—}009522), SEQ.ID.NO.7 (GenBank accession #NM_{—}030753); and/or SEQ.ID.NO.NO.8 (GenBank accesion #NM_{—}033131).

β-catenin polynucleotide sequences include, but are not limited to SEQ.ID.NO.9(GenBank accession #XM_{—}208760). The GSK-3β polypeptide sequences include, but are not limited to, SEQ.ID.NO.10 (GenBank accession #NM_{—}002093).

As used herein, the term “GSK-3” means the enzyme glycogen synthase kinase 3 and its homologs. As discussed herein, GSK-3 is conserved among organisms across the phylogenetic spectrum, although the homologs present in various organisms differ in ways that are not significant for the purposes of the present invention. One of skill in the art will appreciate that the present invention may be practiced using any of the eukaryotic homologs of GSK-3. Furthermore, vertebrate GSK-3 exists in two isoforms, denoted GSK-3α and GSK-3β. GSK-3α and GSK-3β differ from one another only in ways that are not significant for the purposes of the present invention. Therefore, the terms “GSK-3”, “GSK-3α”, and “GSK-3β” are used interchangeably herein. Although the preferred embodiment of the present invention and the examples presented herein exemplify the study and use of GSK-3β, the invention should not be considered to be limited to this particular isoform of GSK-3.

Thus, nucleic acid compositions encoding Wnt, β-catenin, or GSK-3β amino acid sequences are herein provided and are also available to a skilled artisan at accessible databases, including the National Center for Biotechnology Information's GenBank database and/or commercially available databases, such as from Celera Genomics, Inc. (Rockville, Md.). Also included are splice variants that encode different forms of the protein, if applicable. The nucleic acid sequences may be naturally occurring or synthetic.

As used herein, the terms “Wnt, β-catenin, and/or GSK-3β nucleic acid sequence,” “Wnt, β-catenin, and/or GSK-3β polynucleotide,” and “Wnt, β-catenin, and/or GSK-3β gene” refer to nucleic acids provided herein, homologs thereof, and sequences having substantial similarity and function, respectively. A skilled artisan recognizes that the sequences are within the scope of the present invention if they encode a product which regulates at least one of the following functions, activation of the Wnt/β-catenin signaling pathway, activation of Wnt dependent genes, accumulation of β-catenin, inhibition of phosphorylation of β-catenin, cardiac myogenesis, increased expression of cardiac specific transcription factors or genes, and furthermore knows how to obtain such sequences, as is standard in the art.

The term “substantially identical”, when used to define either a Wnt, β-catenin, and/or GSK-3β amino acid sequence or Wnt, β-catenin, and/or GSK-3β polynucleotide sequence, means that a particular subject sequence, for example, a mutant sequence, varies from the sequence of natural Wnt, β-catenin, and/or GSK-3β, respectively, by one or more substitutions, deletions, or additions, the net effect of which is to retain at least some of the biological activity found in the native Wnt, β-catenin, and/or GSK-3β protein, respectively. Alternatively, DNA analog sequences are “substantially identical” to specific DNA sequences disclosed herein if: (a) the DNA analog sequence is derived from coding regions of the natural Wnt, β-catenin, and/or GSK-3β gene, respectively; or (b) the DNA analog sequence is capable of hybridization to DNA sequences of Wnt, β-catenin, and/or GSK-3β under moderately stringent conditions and Wnt, β-catenin, and/or GSK-3β, respectively having biological activity similar to the native proteins; or (c) DNA sequences which are degenerative as a result of the genetic code to the DNA analog sequences defined in (a) or (b). Substantially, identical analog proteins will be greater than about 80% similar to the corresponding sequence of the native protein. Sequences having lesser degrees of similarity but comparable biological activity are considered to be equivalents. In determining polynucleotide sequences, all subject polynucleotide sequences capable of encoding substantially similar amino acid sequences are considered to be substantially similar to a reference polynucleotide sequence, regardless of differences in codon sequence.

As used herein, “hybridization”, “hybridizes” or “capable of hybridizing” is understood to mean the forming of a double or triple stranded molecule or a molecule with partial double or triple stranded nature. The term “hybridization”, “hybridize(s)” or “capable of hybridizing” encompasses the terms “stringent condition(s)” or “high stringency” and the terms “low stringency” or “low stringency condition(s)” or “moderately stringent conditions”.

As used herein “stringent condition(s)” or “high stringency” are those conditions that allow hybridization between or within one or more nucleic acid strand(s) containing complementary sequence(s), but precludes hybridization of random sequences. Stringent conditions tolerate little, if any, mismatch between a nucleic acid and a target strand. Such conditions are well known to those of ordinary skill in the art, and are preferred for applications requiring high selectivity. Non-limiting applications include isolating a nucleic acid, such as a gene or a nucleic acid segment thereof, or detecting at least one specific mRNA transcript or a nucleic acid segment thereof, and the like.

Stringent conditions may comprise low salt and/or high temperature conditions, such as provided by about 0.02 M to about 0.15 M NaCl at temperatures of about 50° C. to about 70° C. It is understood that the temperature and ionic strength of a desired stringency are determined in part by the length of the particular nucleic acid(s), the length and nucleobase content of the target sequence(s), the charge composition of the nucleic acid(s), and to the presence or concentration of formamide, tetramethylammonium chloride or other solvent(s) in a hybridization mixture.

It is also understood that these ranges, compositions and conditions for hybridization are mentioned by way of non-limiting examples only, and that the desired stringency for a particular hybridization reaction is often determined empirically by comparison to one or more positive or negative controls. Depending on the application envisioned, it is preferred to employ varying conditions of hybridization to achieve varying degrees of selectivity of a nucleic acid towards a target sequence. In a non-limiting example, identification or isolation of a related target nucleic acid that does not hybridize to a nucleic acid under stringent conditions may be achieved by hybridization at low temperature and/or high ionic strength. For example, a medium or moderate stringency condition could be provided by about 0.1 to 0.25 M NaCl at temperatures of about 37° C. to about 55° C. Under these conditions, hybridization may occur even though the sequences of probe and target strand are not perfectly complementary, but are mismatched at one or more positions. In another example, a low stringency condition could be provided by about 0.15 M to about 0.9 M salt, at temperatures ranging from about 20° C. to about 55° C. Of course, it is within the skill of one in the art to further modify the low or high stringency conditions to suite a particular application. For example, in other embodiments, hybridization may be achieved under conditions of, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

Other compositions of Wnt modulators can include, but are not limited to compositions discussed in U.S. Pat. Nos. 5,851,984, and 6,159,462 which are incorporated herein by reference in their entirety. Yet further, modulator compositions of β-catenin can include, but are not limited to compositions discussed in U.S. Pat. No. 6,465,249, which is incorporated herein by reference in its entirety. Still further, modulator compositions of GSK-3β can include, but are not limited to compositions discussed in U.S. Pat. No. 6,441,053 in its entirety.

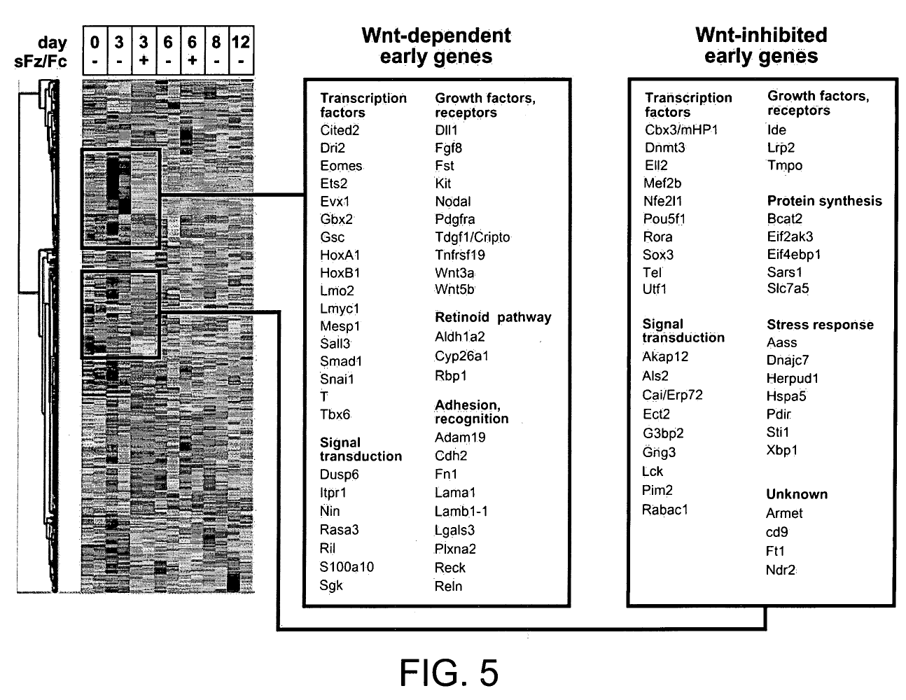

Still further, Wnt modulators can include compositions that modulate Wnt-dependent early genes or Wnt-inhibited early genes that are known or yet to be identified, for example, see FIG. 5.

B. Expression Vectors

The present invention can involve using expression constructs as the pharmaceutical compositions. It is contemplated that the expression construct comprises polynucleotide sequences encoding polypeptides which can act as modulators of Wnt/β-catenin signaling. Such expression constructs include, but are not limited to constructs containing an inhibitor of GSK-3β expression or an activator of Wnt or β-catenin expression or any other modulator that can modulate Wnt/β-catenin signaling. It is contemplated that the inhibitor of GSK-3β modulates or suppresses phosphorylation of β-catenin resulting in an accumulation of β-catenin.

In certain embodiments, the present invention involves the manipulation of genetic material to produce expression constructs that encode inhibitors of GSK-3β or activators of Wnt and/or β-catenin. Thus, the inhibitor or activator is contained in an expression vector. Such methods involve the generation of expression constructs containing, for example, a heterologous nucleic acid sequence encoding an inhibitor or activator of interest and a means for its expression, replicating the vector in an appropriate cell, obtaining viral particles produced therefrom, and infecting cells with the recombinant virus particles.

In one embodiment, a gene encoding a Wnt and/or β-catenin and/or GSK-3β or structural/functional domain thereof is introduced in a viral vector. Such vectors include an attenuated or defective DNA virus, such as but not limited to herpes simplex virus (HSV), papilloma virus, Epstein Barr virus (EBV), adenovirus, adeno-associated virus (AAV), lentivirus and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, any tissue can be specifically targeted. Examples of particular vectors include, but are not limited to, a defective herpes virus 1 (HSV1) vector (Kaplitt et al., 1991) an attenuated adenovirus vector, (Stratford-Perricaudet et al., 1992), and a defective adeno-associated virus vector (Samulski et al., 1987 and Samulski et al., 1989).

Alternatively, the vector can be introduced in vitro by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for transfection of a gene encoding a marker (Felgner et. al., 1987; Mackey et al., 1988). The use of cationic lipids may promote encapsulation of negatively charged nucleic acids, and also promote fusion with negatively charged cell membranes (Felgner and Ringold, 1989).

It is also possible to introduce the vector as a naked DNA plasmid. Naked DNA vectors for gene therapy can be introduced into the desired host cells by methods known in the art, e.g., transfection, electroporation, microinjection, transduction, cell fusion, DEAE dextran, calcium phosphate precipitation, use of a gene gun, or use of a DNA vector transporter (Wu et al., 1992; Wu and Wu, 1988).

C. Transcription Factors and Nuclear Binding Sites

Transcription factors are regulatory proteins that binds to a specific DNA sequence (e.g., promoters and enhancers) and regulate transcription of an encoding DNA region. Typically, a transcription factor comprises a binding domain that binds to DNA (a DNA binding domain) and a regulatory domain that controls transcription. Where a regulatory domain activates transcription, that regulatory domain is designated an activation domain. Where that regulatory domain inhibits transcription, that regulatory domain is designated a repression domain.

Activation domains, and more recently repression domains, have been demonstrated to function as independent, modular components of transcription factors. Activation domains are not typified by a single consensus sequence but instead fall into several discrete classes: for example, acidic domains in GAL4 (Ma, et al., 1987), GCN4 (Hope, et al., 1987), VP16 (Sadowski, et al., 1988), and GATA-1 (Martin, et al., 1990); glutamine-rich stretches in Sp1 (Courey, et al., 1988) and Oct-2/OTF2 (Muller-Immergluck, et al., 1990; Gerster, et al., 1990); proline-rich sequences in CTF/NF-1 (Mermod, et al., 1989); and serine/threonine-rich regions in Pit-1/GH-F-1 (Theill, et al., 1989) all function to activate transcription. The activation domains of fos and jun are rich in both acidic and proline residues (Abate, et al., 1991; Bohmann, et al., 1989); for other activators, like the CCAAT/enhancer-binding protein C/EBP (Friedman, et al., 1990), no evident sequence motif has emerged.

In the present invention, it is contemplated that transcription factors can be used to inhibit the expression of a GSK-3β gene and/or enhance or activate the expression of Wnt and/or β-catenin.

D. Antisense and Ribozymes

An antisense molecule that binds to a translational or transcriptional start site, or splice junctions, are ideal inhibitors. Antisense, ribozyme, and double-stranded RNA molecules target a particular sequence to achieve a reduction or elimination of a particular polypeptide, such as GSK-3β. Thus, it is contemplated that antisense, ribozyme, and double-stranded RNA, and RNA interference molecules are constructed and used to inhibit GSK-3β expression or any other polypeptide in which inhibition of the peptide results in enhancement of the Wnt/β-catenin signaling pathway.
1. Antisense Molecules

Antisense methodology takes advantage of the fact that nucleic acids tend to pair with complementary sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, are employed to inhibit gene transcription or translation or both within a host cell, either in vitro or in vivo, such as within a host animal, including a human subject.

Antisense constructs are designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction are used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

It is advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.
2. Ribozymes

Ribozymes are RNA-protein complexes that cleave nucleic acids in a site-specific fashion. Ribozymes have specific catalytic domains that possess endonuclease activity (Kim and Cech, 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression is particularly suited to therapeutic applications (Scanlon et al., 1991; Sarver et al., 1990; Sioud et al., 1992). Most of this work involved the modification of a target mRNA, based on a specific mutant codon that is cleaved by a specific ribozyme. In light of the information included herein and the knowledge of one of ordinary skill in the art, the preparation and use of additional ribozymes that are specifically targeted to a given gene will now be straightforward.

Other suitable ribozymes include sequences from RNase P with RNA cleavage activity (Yuan et al., 1992; Yuan and Altman, 1994), hairpin ribozyme structures (Berzal-Herranz et al., 1992; Chowrira et al., 1993) and hepatitis δ virus based ribozymes (Perrotta and Been, 1992). The general design and optimization of ribozyme directed RNA cleavage activity has been discussed in detail (Haseloff and Gerlach, 1988; Symons, 1992; Chowrira, et al., 1994; and Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (A,C or U; Perriman, et al., 1992; Thompson, et al., 1995). The frequency of this dinucleotide occurring in any given RNA is statistically 3 out of 16.

Designing and testing ribozymes for efficient cleavage of a target RNA is a process well known to those skilled in the art. Examples of scientific methods for designing and testing ribozymes are described by Chowrira et al., (1994) and Lieber and Strauss (1995), each incorporated by reference. The identification of operative and preferred sequences for use in GSK-3β targeted ribozymes is simply a matter of preparing and testing a given sequence, and is a routinely practiced screening method known to those of skill in the art.

### 3. RNA Interference

It is also contemplated in the present invention that double-stranded RNA is used as an interference molecule, e.g., RNA interference (RNAi). RNA interference is used to “knock down” or inhibit a particular gene of interest by simply injecting, bathing or feeding to the organism of interest the double-stranded RNA molecule. This technique selectively “knock downs” gene function without requiring transfection or recombinant techniques (Giet, 2001; Hammond, 2001; Stein P, et al., 2002; Svoboda P, et al., 2001; Svoboda P, et a., 2000).

Thus, in certain embodiments, double-stranded GSK-3β RNA is synthesized or produced using standard molecular techniques well known and used by those of skill in the art.

E. Protein Variants

Amino acid sequence variants of the Wnt and/or β-catenin and/or GSK-3β proteins can be used as modulators of Wnt and/or β-catenin and/or GSK-3β. These variants can be substitutional, insertional or deletion variants. These variants may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

Substitutional variants or replacement variants typically contain the exchange of one amino acid for another at one or more sites within the protein. Substitutions can be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

It is thus contemplated by the inventors that various changes may be made in the DNA sequences of genes without appreciable loss of their biological utility or activity, as discussed below. The activity being activation of Wnt/β-catenin signaling, activation of Wnt dependent genes, stabilization of β-catenin, cardiac myogenesis, etc.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine −0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtains a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.
1. Fusion Proteins

A specialized kind of insertional variant is the fusion protein. This molecule generally has all or a substantial portion of the native molecule, linked at the N- or C-terminus, to all or a portion of a second polypeptide. For example, a fusion protein of the present invention can includes the addition of a protein transduction domains, for example, but not limited to Antennepedia transduction domain (ANTP), HSV1 (VP22) and HIV-1(Tat) (Ryu et al., 2003; and Mi et al., 2000). Fusion proteins containing protein transduction domains (PTDs) can traverse biological membranes efficiently, thus delivering the protein of interest (Wnt and/or β-catenin and/or GSK-3β or variant thereof, such as an activator or inhibitor) into the cell.

Yet further, inclusion of a cleavage site at or near the fusion junction will facilitate removal of the extraneous polypeptide after purification. Other useful fusions include linking of functional domains, such as active sites from enzymes, glycosylation domains, other cellular targeting signals or transmembrane regions.
2. Domain Switching

An interesting series of variants can be created by substituting homologous regions of various proteins. This is known, in certain contexts, as “domain switching.”

Domain switching involves the generation of chimeric molecules using different but, in this case, related polypeptides. By comparing various Wnt and/or β-catenin and/or GSK-3β proteins, one can make predictions as to the functionally significant regions of these molecules. It is possible, then, to switch related domains of these molecules in an effort to determine the criticality of these regions to function of the protein. These molecules may have additional value in that these “chimeras” can be distinguished from natural molecules, while possibly providing the same function.
3. Synthetic Peptides

The present invention also describes smaller Wnt and/or β-catenin and/or GSK-3β related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Tam et al., (1983); Heath and Merrifield (1986); and Wong and Merrifield (1980), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

### IV. Screening for Modulators

The present invention comprises methods for identifying modulators that affect the activation of the Wnt-catenin signaling pathway. These assays may comprise random screening of large libraries of candidate substances; alternatively, the assays may be used to focus on particular classes of compounds selected with an eye towards structural attributes that are believed to make them more likely to modulate the function or activity of Wnt and/or β-catenin and/or GSK-3β

By function, it is meant that one may assay for mRNA expression, protein expression, protein activity, or ability to associate and/or dissociate from other members of the complex and otherwise determine functions contingent on the Wnt and/or β-catenin and/or GSK-3β proteins.

A. Modulators and Assay Formats

The present invention provides methods of screening for modulators of Wnt and/or β-catenin and/or GSK-3β activity, e.g., activity of Wnt and/or β-catenin and/or GSK-3β and/or expression of Wnt and/or β-catenin and/or GSK-3β.
1. Assay Formats

In one embodiment, the present invention is directed to a method of: obtaining a Wnt and/or β-catenin and/or GSK-3β; contacting the Wnt and/or β-catenin and/or GSK-3β with a candidate substance; and assaying for Wnt and/or β-catenin and/or GSK-3β activity. The difference between the measured activity with and without the candidate substance indicates that said candidate substance is, indeed, a modulator of the Wnt and/or β-catenin and/or GSK-3β activity. Assays may be conducted in cell free systems, in isolated cells, or in organisms including transgenic animals.
2. Inhibitors

An inhibitor according to the present invention may be one which exerts an inhibitory effect on the expression, activity or function of GSK-3β. The inhibitor may inhibit GSK-3β anywhere along its pathway.
3. Activators

An activator according to the present invention may be one which exerts a positive or stimulatory effect on the expression, activity or function of Wnt and/or β-catenin. It is envisioned that the “activator” or “effector” can activate Wnt and/or β-catenin at any point along a pathway, for example, but not limited to increasing expression of cardiac specific genes or increasing activity of cardiac specific proteins. Such cardiac specific genes and/or proteins include, but are not limited to TCF/LEF, NODAl, CRIPTO, Nkx2,5, GATA4, MEF2C, Tbx5, cardiac myosin heavy chains, Wnt-dependent early genes as identified in FIG. 5, etc.
4. Candidate Substance

As used herein, the term “candidate substance” refers to any molecule that may potentially modulate Wnt and/or β-catenin and/or GSK-3β activity, expression or function. Candidate compounds may include fragments or parts of naturally-occurring compounds or may be found as active combinations of known compounds which are otherwise inactive. The candidate substance can be a polynucleotide, a polypeptide, a small molecule, etc. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds.

One basic approach to search for a candidate substance is screening of compound libraries. One may simply acquire, from various commercial sources, small molecule libraries that are believed to meet the basic criteria for useful drugs in an effort to “brute force” the identification of useful compounds. Screening of such libraries, including combinatorially generated libraries, is a rapid and efficient way to screen a large number of related (and unrelated) compounds for activity. Combinatorial approaches also lend themselves to rapid evolution of potential drugs by the creation of second, third and fourth generation compounds modeled of active, but otherwise undesirable compounds. It will be understood that an undesirable compound includes compounds that are typically toxic, but have been modified to reduce the toxicity or compounds that typically have little effect with minimal toxicity and are used in combination with another compound to produce the desired effect.

In specific embodiments, a small molecule library that is created by chemical genetics may be screened to identify a candidate substance that may be a modulator of the present invention (Clemons et al., 2001; Blackwell et al., 2001). Chemical genetics is the technology that uses small molecules to modulate the functions of proteins rapidly and conditionally. The basic approach requires identification of compounds that regulate pathways and bind to proteins with high specificity. Small molecules are prepared using diversity-oriented synthesis, and the split-pool strategy to allow spatial segregation on individual polymer beads. Each bead contains compounds to generate a stock solution that can be used for many biological assays.

The most useful pharmacological compounds may be compounds that are structurally related to compounds which interact naturally with enzymes that bind the telomere. Creating and examining the action of such molecules is known as “rational drug design,” and include making predictions relating to the structure of target molecules. Thus, it is understood that the candidate substance identified by the present invention may be a small molecule activator or any other compound (e.g., polypeptide or polynucleotide) that may be designed through rational drug design starting from known activators of the Wnt/β-catenin signaling pathway.

The goal of rational drug design is to produce structural analogs of biologically active target compounds. By creating such analogs, it is possible to fashion drugs which are more active or stable than the natural molecules, which have different susceptibility to alteration or which may affect the function of various other molecules. In one approach, one would generate a three-dimensional structure for a molecule like Wnt and/or β-catenin and/or GSK-3β or another other protein involved in the Wnt/β-catenin signaling pathway, and then design a molecule for its ability to interact with Wnt and/or β-catenin and/or GSK-3β and/or any other protein involved in the pathway. This could be accomplished by X-ray crystallography, computer modeling or by a combination of both approaches.

It also is possible to use antibodies to ascertain the structure of a target compound or activator. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

It will, of course, be understood that all the screening methods of the present invention are useful in themselves notwithstanding the fact that effective candidates may not be found. The invention provides methods for screening for such candidates, not solely methods of finding them.

B. In vitro Assays

A quick, inexpensive and easy assay to run is a binding assay. Binding of a molecule to a target (e.g., Wnt and/or β-catenin and/or GSK-3β) may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to a Wnt and/or β-catenin and/or GSK-3β molecule or fragment thereof is provided.

A target Wnt and/or β-catenin and/or GSK-3β molecule may be either free in solution, fixed to a support, expressed in or on the surface of a cell. Either the Wnt and/or β-catenin and/or GSK-3β molecule or the compound may be labeled, thereby indicating if binding has occurred. In another embodiment, the assay may measure the activation of binding of a Wnt and/or β-catenin and/or GSK-3β molecule to a natural or artificial substrate or binding partner. Competitive binding assays can be performed in which one of the agents is labeled. Usually, the Wnt and/or β-catenin and/or GSK-3β molecule will be the labeled species, decreasing the chance that the labeling will interfere with the binding moiety's function. One may measure the amount of free label versus bound label to determine binding or activation of binding. These approaches may be utilized on cell cycle checkpoint kinases.

A technique for high throughput screening of compounds is described in WO 84/03564 which is invorporated herein by reference. Large numbers of small peptide test compounds are synthesized on a solid substrate, such as plastic pins or some other surface. The peptide test compounds are reacted with, for example, telomere associated protein and washed. Bound polypeptide is detected by various methods.

C. In Cyto Assays

Various cell lines that express Wnt and/or β-catenin and/or GSK-3β molecule can be utilized for screening of candidate substances. For example, cells containing Wnt and/or β-catenin and/or GSK-3β molecule with an engineered indicator can be used to study various functional attributes of candidate compounds. In such assays, the compound would be formulated appropriately, given its biochemical nature, and contacted with a target cell. This same approach may utilized to study various functional attributes of candidate compounds that effect the Wnt/β-catenin signaling pathway.

Depending on the assay, culture may be required. As discussed above, the cell may then be examined by virtue of a number of different physiologic assays (e.g., growth, size, or survival). Alternatively, molecular analysis may be performed in which the function of Wnt and/or β-catenin and/or GSK-3β molecule and related pathways may be explored. This involves assays such as those for protein production, enzyme function, substrate utilization, mRNA expression (including differential display of whole cell or polyA RNA) and others.

### V. Treatment of Cardiovascular Disease

Embodiments of the present invention include the methods of delivering to a subject a cardiomyocyte cell, generated by enhancing or activating the Wnt/β-catenin signaling pathway to differentiate a non-cardiomyocyte cell to a cardiomyocyte cell, or a pharmaceutical composition comprising the same to treat cardiovascular disease.

Cardiovascular diseases and/or disorders include, but are not limited to, diseases and/or disorders of the pericardium, heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). In specific embodiments, the cardiovascular disease includes, but is not limited to, coronary artery diseases (i.e., arteriosclerosis, atherosclerosis, and other diseases of the arteries, arterioles and capillaries or related complaint), myocardial infarction and ischemic heart disease.

Accordingly, the invention involves the administration of cardiomyocyte cells generated by methods described herein or tissues derived therefrom or a pharmaceutical composition comprising same as a treatment or prevention of any one or more of these conditions or other conditions involving weakness and/or damage in the heart. It is envisioned that one of skill in the art will know the most advantageous routes of administration depending upon the disease. In specific embodiments, it is contemplated that the cardiomyocyte cells or pharmaceutical composition comprising same can be administered via injection, which includes, but is not limited to subcutaneous, intravenous, intraarterial, intramuscular, intraperitoneal, intramyocaridal, transendocardial, transepicardial, intranasal and intrathecal.

Yet further, it is envisioned that the cardiomyocyte cells or pharmaceutical composition of the present invention can be administered to the subject in an injectable formulation containing any compatible carrier, such as various vehicles, adjuvants, additives, and diluents. Yet further, the cardiomyocyte cells or pharmaceutical composition can be administered parenterally to the subject in the form of slow-release subcutaneous implants or targeted delivery systems such as monoclonal antibodies, iontophoretic, polymer matrices, liposomes, and microspheres.

Treatment regimens may vary as well, and often depend on the cardiovascular disease or disorder, disease progression, and health and age of the subject. Obviously, certain types of cardiovascular disease will require more aggressive treatment, while at the same time, certain patients cannot tolerate more taxing protocols. The clinician will be best suited to make such decisions based on the known efficacy and toxicity (if any) of the therapeutic formulations.

Suitable regimes for initial administration and further doses or for sequential administrations also are variable, and may include an initial administration followed by subsequent administrations; but nonetheless, may be ascertained by the clinician.

For example, the cardiomyocyte cells or tissues derived therefrom or the pharmaceutical composition thereof can be administered initially, and thereafter maintained by further administration. For instance, a composition of the invention can be administered in one type of composition and thereafter further administered in a different or the same type of composition. For example, a composition of the invention can be administered by intravenous injection to bring blood levels to a suitable level. The subject's levels are then maintained by a subcutaneous implant form, although other forms of administration, dependent upon the subject's condition, can be used.

As used herein the term “effective amount” is defined as an amount of the cells or pharmaceutical composition thereof that will repair damaged myocardium, regenerate cardiomyocytes, regenerate vascular cells, provide structural stability to an injured myocardium or provide at least partially restored functionality to an injured myocardium. Thus, an effective amount is an amount sufficient to detectably and repeatedly ameliorate, reduce, minimize or limit the extent of the disease or its symptoms.

The precise determination of what would be considered an effective dose may be based on factors individual to each subject, including their size, age, size of the infarct, and amount of time since damage. Therefore, dosages can be readily ascertained by those skilled in the art from this disclosure and the knowledge in the art. Thus, the skilled artisan can readily determine the amount of compound and optional additives, vehicles, and/or carrier in compositions and to be administered in methods of the invention. Of course, for any composition to be administered to an animal or human, and for any particular method of administration, it is preferred to determine the toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. Furthermore, the time for sequential administrations can be ascertained without undue experimentation.

The treatments may include various “unit doses.” Unit dose is defined as containing a predetermined-quantity of the therapeutic composition. The quantity to be administered, and the particular route and formulation, are within the skill of those in the clinical arts. A unit dose need not be administered as a single injection but may comprise continuous infusion over a set period of time.

In further embodiments, the cardiomyocyte cells or tissues derived therefrom are administered to a subject suffering from myocardial infarction. It is contemplated that the differentiated cardiomyocyte cells can alleviate the symptoms associated with myocardial infarction. For example, the injected cells migrate to the infarcted myocardium. The myocytes them assemble into myocardium tissue resulting in repair or regeneration of the infarcted myocardium.

Further embodiments of the present invention involve a method of targeting injured myocardium by delivering to a subject the differentiated cardiomyocyte cells, as described herein, wherein the cells migrate or home and attach to the injured myocardium. The cardiomyocyte cells are administered intravenously to the subject. Thus, the cardiomyocyte cells maneuver the systemic circulation and migrate or target or home to the damaged or injured myocardium. Once the cardiomyocyte cells have migrated to the damaged myocardium, the cardiomyocytes cells repair the damage myocardium. It is envisioned that the administration of the cardiomyocytes will restore both structural and functional integrity to a damaged myocardium. Functional integrity refers to the ability of the cardiomyocyte cells to incorporate into the myocardium and improve functional measures of cardiac output, for example, contractility, pump volume, etc. Contractile strength or contractility can be measured by measuring the maximum rate of change in pressure (dp/dt max). Clinically, contractility is measured by ejection fraction. Normally, the heart ejects about 60% of its volume each beat, thus a increase in the volume is an indicator of increased contractility or contractile strength and ventricular function.

In further embodiments, the present invention involves a method of repairing injured coronary vessels by administering to the subject an effective amount of cardiomyocyte cells generated by methods described herein, such that the amount results in regeneration of coronary vascular cells to repair the coronary vasculature.

Another embodiment is a method of generating cardiomyocyte cells from non-cardiomyocyte cells. The cardiomyocyte cells can be generated in vivo or in vitro. In a specific embodiment, the cardiomyocyte cells are generated in vitro. In generating cardiomyocyte cells, non-cardiomyocyte cells are obtained from a source. The source can be mammalian and can provide, for example, bone marrow, umbilical cord blood, umbilical tissue, circulating endothelial progenitor cells, cardiac fibroblasts, adipose tissue or skin. The non-cardiomyocyte cell obtained from the mammalian source includes, for example, a fibroblast, a stem cell, a progenitor cell. Once the non-cardiomyocyte cells are obtained, they are administered a composition that activates the Wnt/β-catenin signaling pathway, and then they are cultured in vitro so that a plurality of cells is generated.

Yet further, it is also contemplated that additional transcription factor(s) that are useful for cardiac development may be useful in the present invention, for example, Nkx2.5, can be administered to the non-cardiomyocyte cells. The transcription factor can be administered directly to the cultured cells. Yet further, the transcription factor can be administered via an expression vector that expresses the transcription factor. Development of expression vectors are well known and used in the art, for example Manniatis et al., (1982). Once the expression vector is generated, it can be delivered to the cells via standard transfection protocols, which are well-known and used in the art. These standard transfection protocols include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979), cell sonication (Fechheimer et al., 1987), gene bombardment using high velocity microprojectiles (Yang et al., 1990), and receptor-mediated transfection (Wu and Wu, 1987; Wu and Wu, 1988).

Yet further, it is envisioned that the non-cardiomyocyte cells are obtained from an autologous source. The autologous source can be tissue that is obtained from a tissue biopsy. The cells are proliferated in vitro to generate an abundance of the autologous cells. After a suitable number of cells have been proliferated, the composition of the present invention that activates Wnt/β-catenin signaling is introduced to the autologous cells to generate cardiomyocyte cells and, in further embodiments, are administered to the subject, such as via an intravenous injection. In other embodiments the autologous source cardiomyocyte cells are administered to an individual as a tissue, such as a cardiac tissue, including a vessel.

It is well known by those of skill in the art that the use of autologous non-cardiomyocyte cells will reduce and/or eliminate an immune reaction that may occur if allogeneic or xenogeneic non-cardiomyocyte cells are used. Allogeneic or xenogeneic cells are initially recognized by the subject's immune system through antigenic determinants expressed on the surface of the cells. The predominant antigens recognized as “non-self” are major histocompatibility complex class I and class II antigens (MHC class I and class II). However, if non-autologous cells are used, one of skill in the art is aware of the various procedures that may be used to reduce the immune reaction to the cells.

One such procedure that is routinely used to inhibit rejection of transplanted cells by the immune system of the subject is the administration of drugs that suppress the function of the immune system. While drugs, such as cyclophosphamide and cyclosporin, effectively inhibit the actions of the immune system, and thus allow acceptance of the cells, their use can cause generalized, non-specific immunosuppression which leaves the subject susceptible to other disorders such as infection. Additionally, administration of immunsuppressive drugs is often accompanied by other serious side effects such as renal failure hypertension.

Another procedure that is readily available to those of skill in the art is to genetically modify the cells. Such genetic modification includes, for example altering at least one of the surface antigens to decrease the recognition of non-self, for example see U.S. Pat. No. 5,679,340, which is incorporated herein by reference. Further modifications can also include packaging of the cells in a liposome, a micelle or other vehicle to mask the cells from the immune system. Thus, one of skill in the art is cognizant of various procedures and techniques that are available to alter a composition so that it is not recognized as “non-self”, thus decreasing the immune response to allogeneic or xenogeneic cell transplantation.

### VI. Combined Cardiac Disease Treatments

In order to increase the effectiveness of the cardiomyocyte cells generated by methods described herein, it may be desirable to combine these compositions and methods of the invention with a known agent effective in the treatment of cardiac disease or disorder. In some embodiments, it is contemplated that a conventional therapy or agent, including but not limited to, a pharmacological therapeutic agent, a surgical therapeutic agent (e.g., a surgical procedure) or a combination thereof, may be combined with the smooth muscle cells of the present invention or a tissue derived therefrom. In a non-limiting example, a therapeutic benefit comprises repair of myocardium or vascular tissue or reduced restenosis following vascular or cardiovascular intervention, such as occurs during a medical or surgical procedure.

This process may involve contacting the cell(s) with an agent(s) and the smooth muscle cells of the present invention, or a tissue derived therefrom, at the same time or within a period of time wherein separate administration of the smooth muscle cells and an agent to a cell, tissue or organism produces a desired therapeutic benefit. The terms “contacted” and “exposed,” when applied to a cell, tissue or organism, are used herein to describe the process by which the smooth muscle cells and/or therapeutic agent(s) are delivered to a target cell, tissue or organism or are placed in direct juxtaposition with the target cell, tissue or organism. The cell, tissue or organism may be contacted (e.g., by administration) with a single composition or pharmacological formulation that comprises both smooth muscle cells and one or more agents, or by contacting the cell with two or more distinct compositions or formulations, wherein one composition includes the cardiomyocyte cells and the other includes one or more agents.

The treatment may precede, be co-current with and/or follow the other agent(s) by intervals ranging from minutes to weeks. In embodiments where the cardiomyocyte cells, and other agent(s) are applied separately to a cell, tissue or organism, one would generally ensure that a significant period of time did not expire between the time of each delivery, such that the cardiomyocyte cells and agent(s) would still be able to exert an advantageously combined effect on the cell, tissue or organism. For example, in such instances, it is contemplated that one may contact the cell, tissue or organism with two, three, four or more modalities substantially simultaneously (i.e. within less than about a minute) as the cardiomyocyte cells or tissue derived therefrom. In other aspects, one or more agents may be administered within of from substantially simultaneously, about minutes to hours to days to weeks and any range derivable therein, prior to and/or after administering the smooth cells or a tissue derived therefrom.

Administration of the cardiomyocyte cell composition to a cell, tissue or organism may follow general protocols for the administration of vascular or cardiovascular therapeutics, taking into account the toxicity, if any. It is expected that the treatment cycles would be repeated as necessary. In particular embodiments, it is contemplated that various additional agents may be applied in any combination with the present invention.

A. Pharmacological Therapeutic Agents

Pharmacological therapeutic agents and methods of administration, dosages, etc., are well-known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, or a vasopressor. Other drug therapies include treatment agents for congestive heart failure, for example, but not limited to calcium channel blocking agents, β-adrenergic blocking agents, angiotensin II inhibitors or ACE inhibitors. ACE inhibitors include drugs designated by the trademarks Accupril®, Altace®, Capoten®, Lotensin®, Monopril®, Prinivil®, Vasotec®, and Zestril®.

B. Surgical Therapeutic Agents

In certain aspects, a therapeutic agent may comprise a surgery of some type, which includes, for example, preventative, diagnostic or staging curative and/or palliative surgery. Surgery, and in particular, a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

Further treatment of the area of surgery may be accomplished by perfusion, direct injection, systemic injection or local application of the area with at least one additional therapeutic agent (e.g., the cardiomyocyte cells of the present invention and/or a tissue derived therefrom, a pharmacological therapeutic agent, and so forth), as would be known to one of skill in the art or described herein.

### VII. EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Differentiation and Transfection

Cells were grown on 10 cm dishes in α-minimal MEM; Invitrogen) supplemented with 10% fetal bovine serum (FBS) (Hyclone), streptomycin. To induce differentiation, cells were seeded at a 1:40 dilution with α-MEM, DMSO. For each experiment, cardiomyocyte differentiation was apparent in the control spontaneous beating, starting day 9 to 10. To obtain stable transformants incorporating GSK-vector control, cells were transfected using Lipofectamine2000 (Invitrogen) and medium containing 500 μg/ml Geniticin (Invitrogen). After 10-14 day, 60 colonies were picked and screened by RT-PCR. To detect exogenous GSK-3β selectively, the 5′ primer corresponded to the N terminus and the 3′ primer to the H. influenza hemagglutinin epitope tag.

### Example 2

### Reverse-Transcriptase Polymerase Chain Reaction (RT-PCR)

RNA was isolated using TRIzol (Invitrogen). RNA (0.1 μg) was subjected to quantitative RT-PCR using the TaqMan One-step RT-PCR Master Mix Biosystems); the RT and PCR were run sequentially using a 7700 Sequence Detector (Applied Biosystems). Copy number for each transcript was expressed relative to that of glyceraldehyde-3-phosphate dehydrogenase (GAPDH), used as a constitutive control. RT-PCR for FGF8 and β-actin was done as described by Lee et al., 2000).

### Example 3

### Immunocytochemistry

Cells were seeded on glass cover slips and cultured with or without DMSO for 3 days. Cells were fixed with 4% paraformaldehyde and penneablized with 0.2% Triton X-100 for 5 min. To detect phosphorylated β-catenin, cells were incubated overnight with rabbit antibody to phoshpo-β-catenin (Ser33/Ser37/Thr4l; Cell Signaling) in Tris-buffered saline, 3% bovine serum albumin, then for 1 hr with goat antibody to rabbit IgG conjugated with Alexa Fluor 488 (Molecular Probes). Immunostaining for sarcomeric myosin heavy chains (MHC) was performed using fluorescein isothiocyanate-conjugated MF20 antibody (Oh et al., 2001). Nuclei were counterstained with DAPI.

### Example 4

### Western Blot Analysis

Cells were seeded on 6-well dishes (1.67×10⁶ cells/well) and cultivated with or without 1% DMSO and 500 ng/ml Fx/FC chimeric protein. After 3 days, cells were harvested in phosphate-buffered saline in 4 C, centrifuged, and resuspended in 20 mM Tris-HCl, pH 7.5, 25 mM sodium fluoride, 1 mM EDTA, containing a protease inhibitor cocktail (Roche Molecular Biochemicals). Cells were incubated on ice for 20 min, followed by 30 strokes in a Dounce homogenizer, and centrifuged at 100,000×g for 30 min. The supernatant was collected as the soluble, cytoplasmic fraction and electrophoresised in 10% SDS-polyacrylamide gels. Proteins were transferred to polyvinylidene which were incubated sequentially in Tris-buffered saline, 2% bovine serum albumin (BSA), 0.05% Tween-20, then mouse antibody to β-catenin (B-D Biosciences) or goat antibody to total actin (C-11; Santa Cruz), overnight at 4° C. Bound antibody was visualized using horseradish-peroxidase (HRP) conjugated goat antibody to mouse IgG (Zymed Laboratories) or donkey antibody to goat IgG enhanced chemiluminescence reagents (Amersham Pharmacia Biotech). To detect phosphorylated β-catenin, cells were lysed with RIPA buffer with 10 nM calyculin A, 10 nM okadaic acid inhibitor cocktail; Western blotting was done as above, using rabbit antibody to phosphoβ-conjugated goat antibody to rabbit IgG (Santa Cruz).

### Example 5

### Luciferase Assays

Cells were seeded and cultured as above and transfected 1 day after plating, using in serum-free αMEM for 6 hr. Transfections contained 0.5 μg TOPFLASH or 0.1 μg pRL-CMV as the co-transfected control. Medium containing 10% FBS with or without DMSO changed 6 and 48 hr after transfection. Cells were lysed 4 days after DMSO treatment and luciferase assayed using the Dual-Luciferase system (Promega). Firefly luciferase activity, indicating TCF-dependent transcription, was normalized to the Renilla luciferase activity of each extract. TOPFLASH activity induced by Wnt3A CM versus control CM was measured after 18 hr. Activity of the GSK-3βA9 expression vector was corroborated by transient transfection in 293T cells: cells were passaged at a 1:2.5 dilution into 24-well dishes, and TOPFLASH, pRL-CMV, and PGK-Wnt3A were co-transfected by calcium-phosphate precipitation along with pcDNA3-GSK-3βA9-HA versus the empty vector control (Jordan et al., 1996). Twenty-four hrs after transfection, the medium was changed to Dulbecco's modified Eagle's medium (DMEM) supplemented with 0.2% BSA. Forty-eight hrs after transfection, cells were lysed and luciferase activities were assayed.

### Example 6

### Induction of Wnt3A and Wnt8A are Early Events in Cell Differentiation

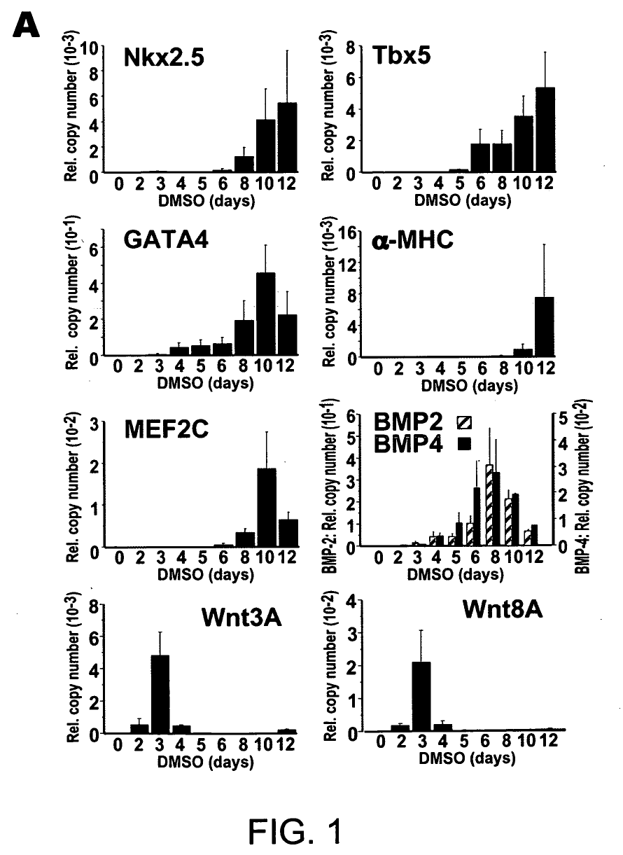

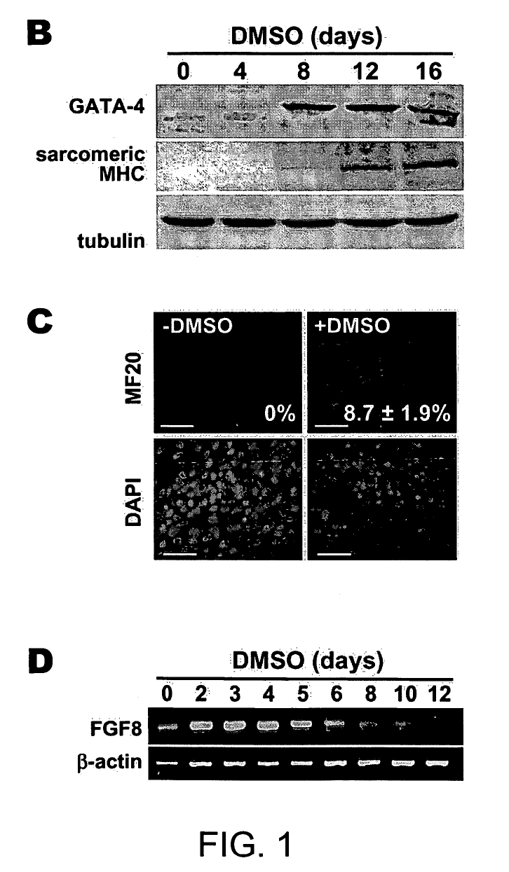

Analyses were used to map the temporal changes of cardiac-specific genes in differentiating comparison to their potential regulators (FIG. 1A). P19C16 ells were cultivated with or without 1% DMSO. Without DMSO, the cells did not express any of the cardiac-specific genes assayed as determined by QRT-PCR. Upon 1% DMSO treatment, the cells expressed the factors Nkx2.5, GATA4, MEF2C, and Tbx5 as early stage markers within 3-8 days. Spontaneous beating was visible at 9 to 10 days, and continued for 4 to 8 days. Among these markers, GATA4 expressed earliest (as early as 3 days after DMSO), and GATA4 protein was detected at 8day (FIG. 1B) as determined by Western blot analysis. α-MHC was seen as early as day 8 and increased until day 12, corresponding to the onset of spontaneous beating.

Next, immunocytochemistry was performed to determine induction of saromeric MHC. Cells were cultivated for 12 days with (right) or without (left) 1% DMSO, and stained with MF20 and DAPI. Concordant with the presence of α-MHC mRNA (FIG. 1A) and protein (FIG. 1B), MF20-positive differentiated myocytes were observed on day 12 (FIG. 1C).

Neither BMP2 nor BMP4 was expressed in the absence of DMSO. Both were induced to low levels within 4 to 5 days, with expression highest on day 8 and persisting through day 12 (FIG. 1A). Previous studies have implicated endogenous BMPs as essential for cardiogenesis in these cells (Monzen et al., 1999; Monzen et al., 2001). By contrast to the delayed and sustained expression of BMP2 and BMP4, Wnt3A and Wnt8A were each induced as early as 2 days after DMSO treatment, with expression greatest on day 3 (FIG. 1A). Thereafter, both Wnt3A and Wnt8A mRNA levels were quickly down-regulated on day 4, with low or undetectable expression at all later times. Like Wnt3A and -8A, FGF8 was strongly induced at 2-4 day and decreased progressively thereafter (FIG. 1C). Wnt11, which was required for Xenopus cardiogenesis (Pandur et al., 2002), was not expressed.

### Example 7

### Wnt/β3-Catenin Pathway is Activated at the Early Stage of Differentiation

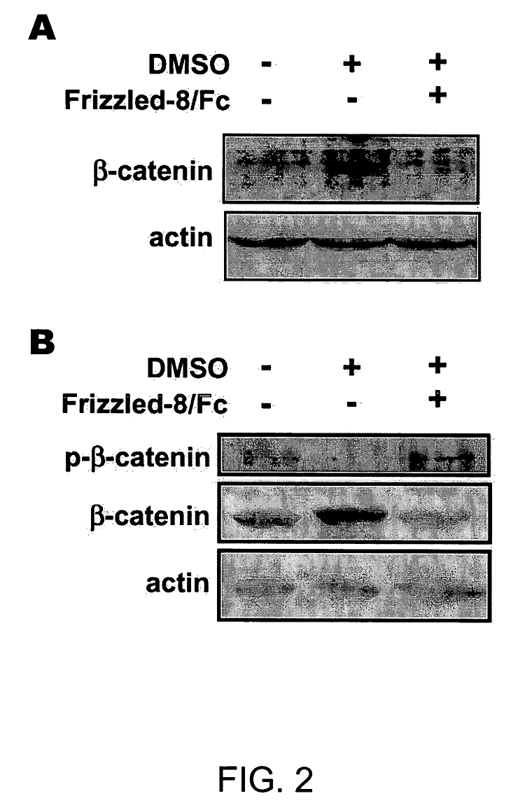

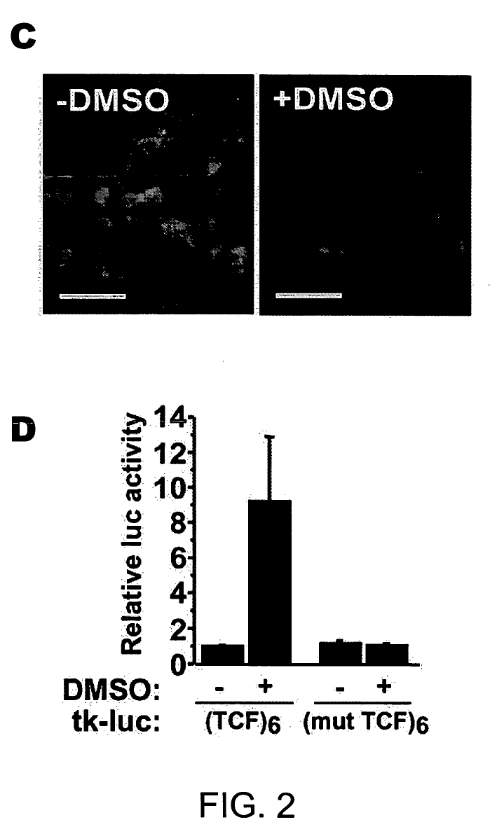

To assess whether or not functional Wnt signaling was activated at the time of Wnt induction, cytosolic β-catenin was examined, which is the crux of the canonical Wnt signal transmission pathway (Moon et al., 2002; Papkoff et al., 1996). Cells treated with or without DMSO were harvested and lysed on day 3, cytosolic protein was fractionated, and β-catenin protein levels were examined by Western blot (FIG. 2A). DMSO caused the accumulation of soluble β-catenin; accumulation of β-catenin was also observed in the total cell lysates (FIG. 2B). Simultaneous treatment with 500 ng/ml Fz-8/Fc chimeric protein, an antagonist for Wnt8A and potentially for other Wnts, decreased β-catenin to the basal level (FIG. 2A, B), indicating that its accumulation was regulated by an autocrine or paracrine circuit in this system, involving endogenous Wnts. Conversely, phosphorylated β-catenin (the form targeted for degradation) was decreased by DMSO treatment for 3 days, determined by immunoblotting and immunocytochemistry with antibody to the phosphorylated epitope (FIG. 2B, FIG. 2C). Furthermore, phosphorylated β-catenin was rescued by treatment with Fz-8/Fc (FIG. 2B). As a third criterion to confirm the activation of the canonical Wnt pathway, TCF-dependent transcriptional activity was measured (FIG. 2D). DMSO treatment for 4 days provoked a 9-fold increase in luciferase activity. No activation was seen using the corresponding control reporter with mutated TCF binding sites. Thus, Wnt induction by DMSO was functionally coupled to activation of the β-catenin pathway.

### Example 8

### The Wnt/β-Catenin Signaling Pathway Was Required for Cardiac Differentiation

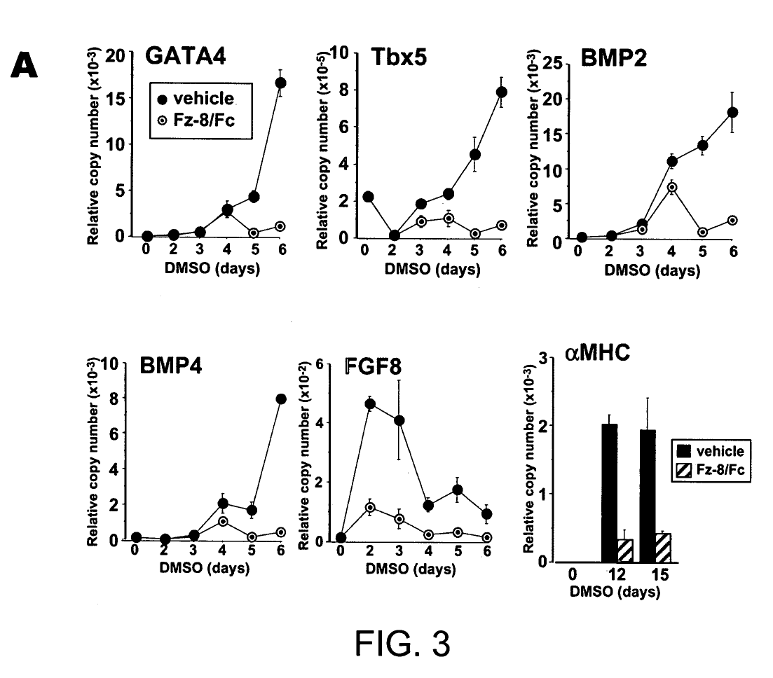

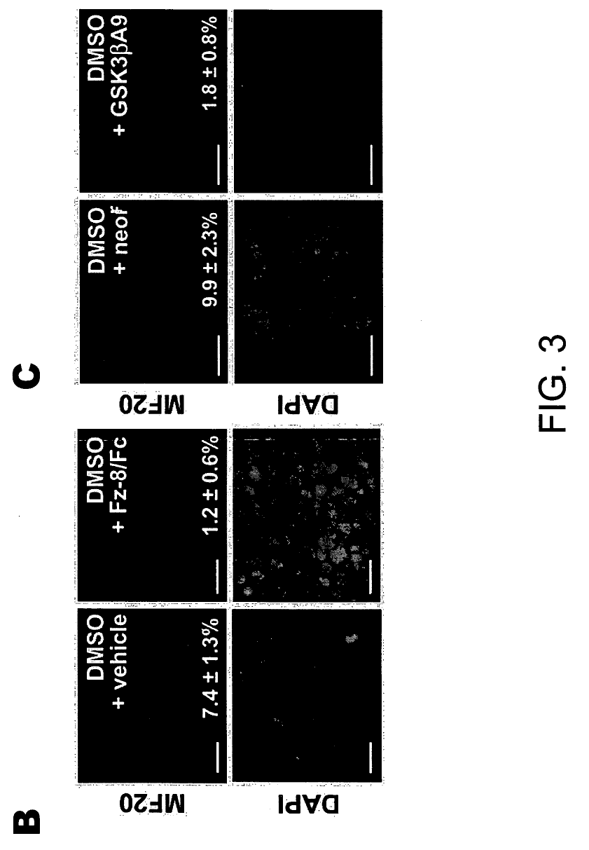

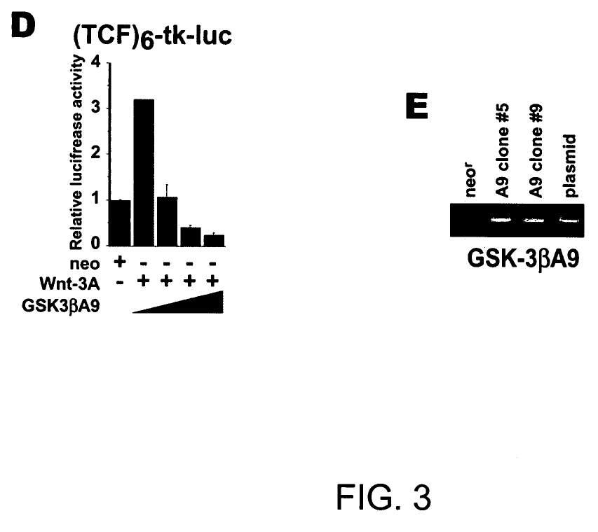

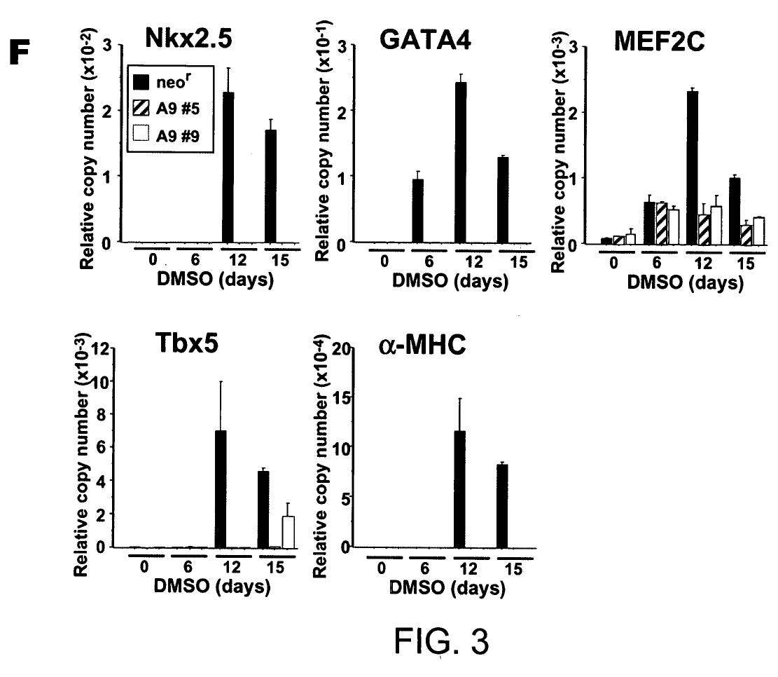

To explore the possible role of Wnt-mediated signaling in early cardiac myogenesis, cell differentiation induced by DMSO was monitored, with and without 200 ng/mlFz Fz-8/Fc. Treatment with the soluble Wnt inhibitor prevented GATA4 and Tbx5 induction by DMSO, at least through day 6 (FIG. 3A). Likewise, Fz-8/Fc inhibited the expression of BMP2, BMP4, and FGF8. Thus, the Wnt pathway lies upstream to the induction of these three cardiac differentiation factors (FIG. 3A). Similar results were obtained using FZ-4/Fc. Correspondingly, Fz-8/Fc decreased the proportion of MF20-positive cells 6- to 7-fold (day 12; FIG. 3B), and suppression continued for at least 15 day.

To determine more specifically whether the β-catenin pathway for Wnt signaling was responsible for Wnt-dependent cardiac myogenesis, a constitutively active form of GSK-3b (pcDNA3-GSK-3βA9-HA) was used. In pilot studies by transient co-transfection of 293T cells (FIG. 3D), it was confirmed that this vector inhibited Wnt3A-induced transcription of the TCF reporter gene. Stable transformants of P19CL6 cells harboring GSK-3bA9 versus the control vector, neo^{r} (FIG. 3E) were obtained. GSK-3βA9 suppressed the induction of cardiac transcription factors by DMSO through at least 15 day, with identical results in independent lines (FIG. 3F). By contrast, cardiac differentiation was impaired in none of the clonal isolates bearing the selectable marker neo alone, assayed at equivalent passage number. Likewise, GSK-3β suppressed the prevalence of sarcomeric MHC staining, whereas transfection with neo had no effect (FIG. 3C). Together, these findings with soluble Fz proteins and activated GSK-3 implicated signaling by endogenous Wnts, via β-catenin, as required for early cardiac determination in this setting.

### Example 9

### The Wnt/β-Catenin Signaling Pathway Enhances Cardiac Myogenesis

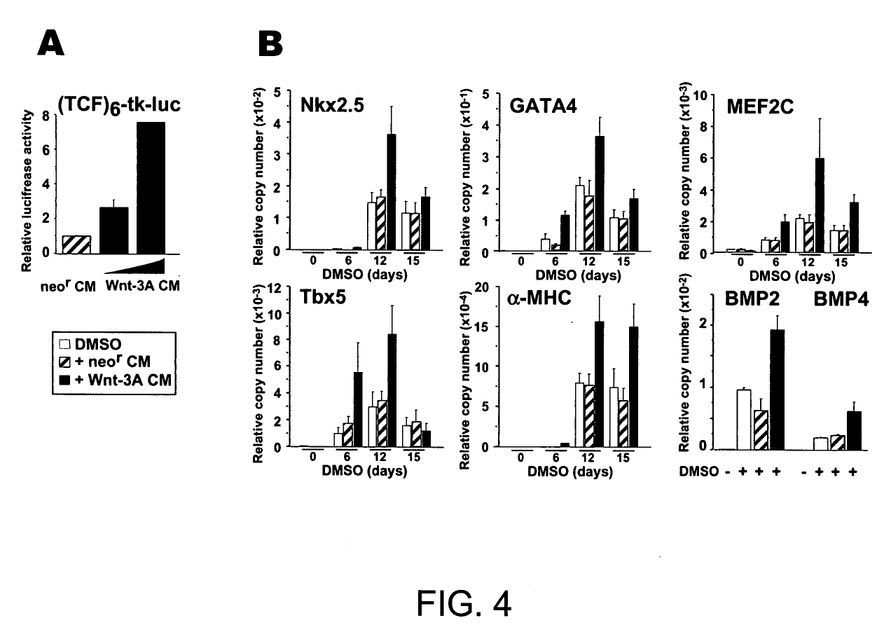

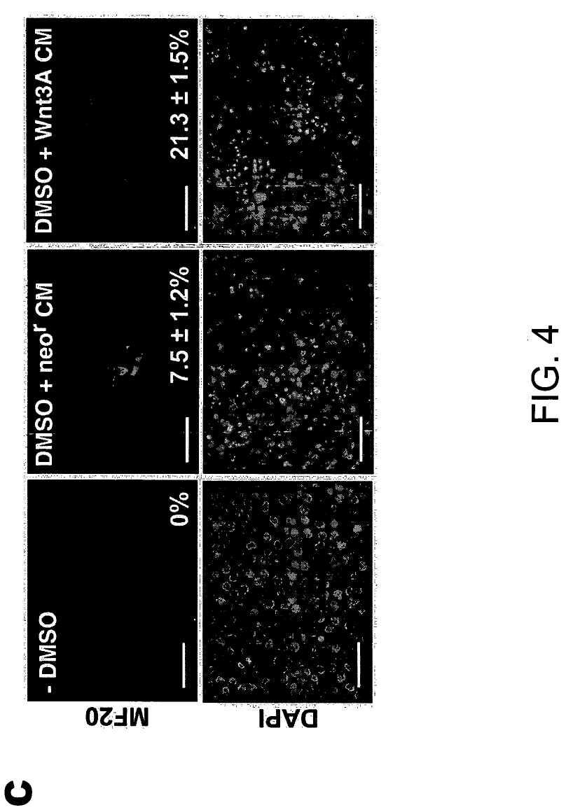

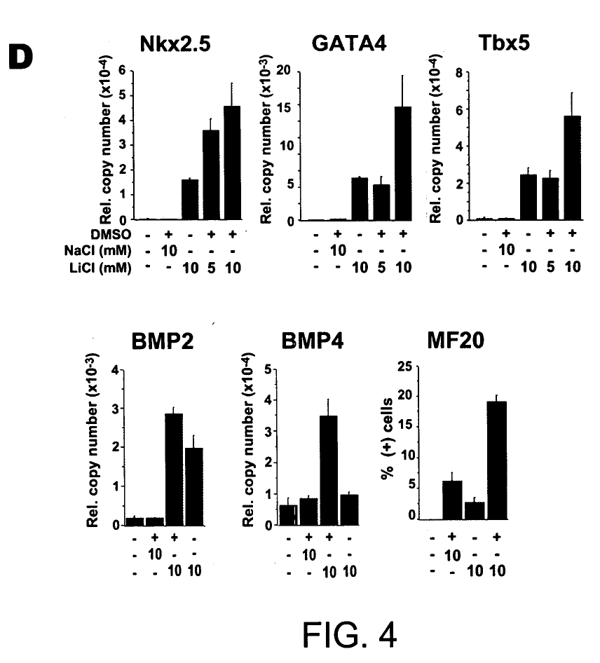

Next, the reciprocal possibility of promoting cardiac differentiation in this system by supplying exogenous Wnt or potentiating β-catenin was explored. Firstly, it was confirmed that Wnt3A-conditioned medium activated TCF-dependent transcription in P19CL6 cells (FIG. 4A). Although control conditioned medium had no effect (from L cells stably transfected with just the neomycin resistance gene), Wnt3A-conditioned medium markedly enhanced the inductive effect of DMSO on Nkx2.5, GATA4, MEF2C and Tbx5 (FIG. 4B). α-MHC, otherwise expressed no sooner than day 8, was detected on day 6 in cultures receiving Wnt3A CM.

Interestingly, Wnt3A CM also enhanced the expression of both BMP2 and BMP4. In accordance with the enhanced expression of cardiogenic factors, the prevalence of MF20-positive cells was increased nearly 3-fold on day 12 by Wnt3A CM (FIG. 4C).

Next, LiCl, which binds and inhibits GSK-3β, was used to Wnt signaling selectively via the β-catenin/TCF pathway (Moon et al., 2002; Chen et al., 2000). At 10 μM, LiCl significantly increased the expression of Nkx2.5, GATA4, Tbx5, BMP2, BMP4 at d 5 and MF20-positive cells at day 12 (FIG. 4D). LiCl itself had an inductive effect on each of the early cardiac markers and on sarcomeric MHC even in the absence of DMSO. However, cells treated with LiCl or Wnt3A CM alone did not show spontaneous beating, suggesting that additional signals conferred by DMSO were necessary for terminal differentiation, beyond just induction of the factors shown here.

### Example 10

### Identification of Wnt Related Genes

P19C16 cells (grown in the presence “+” or absence “−” of a specific extracellular Wnt inhibitor, sFz/Fc), using Affymetrix microarrays to canvas ˜12,000 genes expression. FIG. 5 shows the Wnt-dependent early genes and the Wnt-inhibited early genes that were identified. Any or all of the changes indicated can be involved in steps for the undifferentiated pluripotent cells switch to become cardiac muscle, and become highly relevant candidates to test in heart-forming systems (Sca1 cells, mouse ES cells, human ES cells, human endothelial progenitor cells, etc.). Thus, any or all of these genes and/or proteins (and of agents that alter their expression) can be used as surrogates for Wnts or Wnt signaling proteins or compositions to modulate the Wnt/β-catenin signaling pathway.

### REFERENCES

All patents and publications mentioned in the specifications are indicative of the levels of those skilled in the art to which the invention pertains. All patents and publications are herein incorporated by reference to the same extent as if each individual publication was specifically and individually indicated to be incorporated by reference.
U.S. Pat. No. 4,554,101
U.S. Pat. No. 5,354,855
U.S. Pat. No. 5,679,340
U.S. Pat. No. 5,851,984
U.S. Pat. No. 6,159,462
U.S. Pat. No. 6,441,053
U.S. Pat. No. 6,465,249
Abate et al., Mol Cell Biol. 1991; 11, 3624-32
Aberle et al., (1997) EMBO J 16(13):3797-804.
Alsan et al., Development. 2002; 129, 1935-43
Anisimov et al., Mech. Dev. 2002; 117, 25
Arias et al., Curr. Opin. Genet. Develop. 1999; 9, 447-454
Bejsovec et al., Curr. Biol. 1999; 9, R684-7
Berzal-Herranz et al, (1992)Genes Dev.;6,129-34.
Blackwell et al., Chem Biol. 2001; 8:1167-82.
Bohmann et al., Cell.; Nov. 17, 1989;59(4),709-17
Cech et al., Cell. 1981; 27(3 Pt 2),487-96.
Chen and Okayama, Mol Cell Biol., 1987, 7:2745-52.
Chen et al., J. Biol. Chem. 2000; 275, 17894-9
Chiu et al., Ann Thorac Surg. 60:12-8 (1995).
Chowrira et al, J Biol Chem. 1994; 269,25856-64
Chowrira et al., J Biol Chem.; Sep. 15, 1993;268(26),19458-62
Clemons et al., Chem Biol. 2001; 8:1183-95.
Courey et al., (1988) Cell.;55(5),887-98.
Felgner and Ringold, Science. 1989; 337, 387-388
Felgner et. al., Proc. Natl. Acad. Sci. US.A. 1987; 84, 7413-7417
Forster and Symons, Cell. 1987; 50, 9-16
Fraleyetal., Proc Nat'l Acad Sci. USA. 1979; 76, 3348-3352
Frank et al., Development. 2002; 129, 4591-4603
Frasch et al., Nature. 1995; 374, 464-467
Friedman et al., Genes Dev. 1990; 4, 1416-26
Gerster et al., EMBO J. 1990; 9, 1635-43
Giet et al, J Cell Biol. 2001;152,669-82
Gopal et al., Mol. Cell Biol., 1985; 5,1188-1190
Graham and van der Eb, Virology. 1973; 52, 456-467
Haegel et al., Development. 1995; 121, 3529-37
Hammond et al., Nat Rev Genet. 2001; 2,110-9
Harland and Weintraub, J. Cell Biol., 1985; 101,1094-1099
Haseloff and Gerlach (1988) Nature.;334,585-91.
Heath and Merrifield, Proc Natl Acad Sci USA. 1986; 83:6367-71.
Hiroi et al., Nat. Genet. 2001; 28, 276-80
Hope et al., EMBO J. 1987. 6(9),2781-4
Jackson et al., J. Clin. Invest. 2001; 107, 1395-1402
Jordan et al., Nucleic Acids Res. 1996; 24, 596-601
Joyce et al., Nature. 1989; 338, 217-244
Kaplitt et al., Molec. Cell. Neurosci., 1991; 2:320-330
Kerjanc, Trends Cardiovasc Med. 1999; 9, 139-43
Kim and Cech, Proc Natl Acad Sci USA. 1987; 84(24),8788-92
Koh, G. Y., et al., (1995) J. Clin. Invest. 96, 2034-2042.
Kyte and Doolittle, J Mol Biol. 1982; 157:105-32.
Lee et al., Development. 2000; 127, 5497-508
Lee et al., Nat Biotechnol. 2000; 18, 675-9
Li et al., Circ Res. 78:283-8 (1996).
Li et al., J Thorac Cardiovasc Surg. 119(1):62-8 (2000).
Lickert et al., Dev. Cell. 2002; 3, 171-81
Lieber et al., Mol Cell Biol. 1995; 15,540-51
Logan et al., Development. 1993; 118, 865-875
Ma et al., (1987) 50,137-42
Mackey et al., Proc. Natl. Acad. Sci. USA., 1988; 85:8027-8031
Martin et al., Genes Dev.; 1990; 4,1886-98
Matzuk et al., Nature. 1995; 374, 356-360
Mermod et al., Cell. 1989; 58,741-53
Mi et al., Mol Ther. 2000 ;2:339-47.
Michel and Westhof, J. Mol. Biol. 1990; 216,585-610
Miyoshi et al., (1998) Cancer Res 58(12):2524-7.
Monzen et al., J. Cell. Biol. 2001; 153, 687-98
Monzen et al., Mol. Cell. Biol. 1999; 19, 7096-7105
Moon et al., Science. 2002; 296, 1644-6
Morin et al., (1997) Science 275(5307):1787-90.
Muller et al., (1998) Genes Chromosomes Cancer 22(1):37-41.
Muller-Immergluck et al., EMBO J. 1990; 9,1625-34
Muroyama et al., Genes Dev. 2002; 16, 548-553
Nicolau and Sene, Biochim. Biophys. Acta, 1982; 721,185-190
Oh, et al., Proc. Natl. Acad. Sci. U.S.A. 2001; 98, 10308-10313
Ohteki et al., J. Exp. Med. 2000; 192, 99-104
Palacios et al., (1998) Cancer Res 58(7):1344-7.
Pandur et al., Nature. 2002; 418, 636-41
Papkoffet al., Mol. Cell. Biol. 1996; 16, 2128-2134
Paquin et al., Proc. Natl. Acad. Sci. U.S.A. 2002; 99, 9550-5
Park et al., Dev. Biol. 1996; 177, 104-116
Peng et al., Physiol. Genomics 2002; 9, 145-55
Perriman et al., Gene. 1992; 113:157-63.
Potter et al., Proc. Nat'l Acad. Sci. USA. 1984; 81,7161-7165
Reinhold-Hurek and Shub, Nature, 1992; 357,173-176
Rippe et al., Mol Cell Biol. 10(2):689-95 (1990).
Ryu et al., Mol Cells. 2003; 16:385-91.
Sadowski et al., Nature. 1988; 335,563-4
Samulski et al., J. Virol., 1989; 63:3822-3828
Samulski et al., J. Virol. 1987; 61, 3096-3101
Sarver et al., Science. 1990; 247,1222-1225
Scanlon et. al., Proc. Nat'l Acad. Sci. USA. 1991; 88, 10591-10595
Scorsin et al., Circulation 94(9 Suppl):II337-40 (1996).
Scorsin et al., J Thorac Cardiovasc Surg. 119:1169-75 (2000).
Sioud et al., J Mol Biol. 1992; 223,831-5
Smith et al., J. Cell. Physiol. 1987; 131, 74-84
Soonpaa, et al., Am. J. Physiol. 1996; 271, H2183-H2189
Stein et al., Biochem Biophys Res Commun. 291:1119-22 (2002).
Stratford-Perricaudet et al., J. Clin. Invest., 1992; 90:626-630.
Svoboda et al., Biochem Biophys Res Commun. 287(5):1099-104 (2001).
Svoboda et al., Development. 127(19):4147-56 (2000).
Symons et al., Annu Rev Biochem. 1992;61:641-71.
Tam et al., Int J Pept Protein Res. 1983; 21:57-65.
Tavazzi et al., Eur Heart J. 19 Suppl L:L33-5 (1998).
Theill et al., Nature. 1989; 342, 945-8
Thompson et al., Nucleic Acids Res. 1995; 23:2259-68.
Tomita et al., Circulation. 100(19 Suppl):II247-56 (1999).
Tur-Kaspa et al., Mol. Cell Biol., 1986; 6, 716-718
Voeller et al., (1998) Cancer Res. 58, 2520-2526.
Wong and Merrifield, Biochemistry. 1980; 19:3233-8.
Wu and Wu, Biochem., 1988; 27, 887-892
Wu and Wu, J. Biol. Chem., 1987; 262, 4429-4432
Wu et al., Dev. Biol. 1995; 169, 619-28
Yang et al., Proc. Nat'l Acad. Sci. USA, 1990; 87, 9568-9572
Yuan and Altman Science, 1994; 263, 1269-73
Yuan et al., Proc Natl Acad Sci USA. 1992; 89, 8006-10
Zurawel et al., (1998) Cancer Res. 58, 896-899.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of upregulating expression of a cardiac specific polynucleotide in a cell, comprising the step of delivering a composition that activates Wnt/β-catenin signaling.

2. The method of claim 1, wherein the cell is in a tissue.

3. The method of claim 1, wherein the tissue is in a mammal.

4. The method of claim 3, wherein the mammal is a human.

5. The method of claim 1, wherein said cardiac specific polynucleotide is selected from the group consisting of Nkx2.5, GATA4, MEF2C, Tbx5, CRIPTO, NODAL, and cardiac myosin heavy chain.

6. The method of claim 1, wherein said cell exhibits spontaneous cell beating.

7. The method of claim 1, wherein the composition is a modulator of Wnt.

8. The method of claim 7, wherein the modulator enhances expression of Wnt.

9. The method of claim 7, wherein the modulator enhances activity of Wnt.

10. The method of claim 1, wherein the composition delivers Wnt to the cell.

11. The method of claim 10, wherein Wnt is delivered as a polynucleotide to the cell.

12. The method of claim 1, wherein the composition delivers Wnt as a polypeptide to the cell.

13. The method of claim 1, wherein the composition is a modulator of β-catenin.

14. The method of claim 13, wherein the modulator enhances accumulation of β-catenin.

15. The method of claim 13, wherein the composition inhibits phosphorylation of β-catenin.

16. The method of claim 15, wherein the composition is an inhibitor of glycogen synthase kinase 3β.

17. The method of claim 16, wherein the composition is lithium.

18. A method of upregulating expression of a cardiac specific polynucleotide in a cell, comprising the step of delivering Wnt to the cell.

19. The method of claim 18, wherein said cardiac specific polynucleotide is selected from the group consisting of Nkx2.5, GATA4, MEF2C, Tbx5, CRIPTO, NODAL, and cardiac myosin heavy chain.

20. The method of claim 18, wherein said cell exhibits spontaneous cell beating.

21. The method of claim 18, wherein Wnt is delivered as a polypeptide to the cell.

22. The method of claim 18, wherein Wnt is delivered as a polynucleotide to the cell.

23. The method of claim 22, wherein the polynucleotide is in a vector.

24. The method of claim 23, wherein the vector is a viral vector.

25. The method of claim 23, wherein the vector is a non-viral vector.

26. The method of claim 24, wherein said viral vector is an adenoviral vector, an adeno-associated vector, a retroviral vector or a lentiviral vector.

27. The method of claim 18, wherein the cell is in a tissue.

28. The method of claim 27, wherein the tissue is in a mammal.

29. The method of claim 28, wherein the mammal is a human.

30. A method of enhancing proliferation or differentiation of a cardiomyocyte cell from a non-cardiomyocyte cell, comprising the step of delivering a composition to said non-cardiomyocyte cell that activates Wnt/β-catenin signaling.

31. The method of claim 30, wherein said non-cardiomyocyte cell is derived from autologous tissue.

32. The method of claim 30, wherein said non-cardiomyocyte cell is derived from allogeneic tissue.

33. The method of claim 30, wherein said non-cardiomyocyte cell is derived from xenogeneic tissue.

34. The method of claim 30, wherein said cardiomyocyte cell is defined as a cell comprising at least one of the following:
expression of Nkx2.5;
expression of GATA4;
expression of Tbx5;
expression of MEF2C; and
expression of cardiac myosin heavy chain.

35. The method of claim 30, wherein said cardiomyocyte cell exhibits spontaneous cell beating.

36. The method of claim 30, wherein said non-cardiomyocyte cell is a fibroblast, a stem cell, a progenitor cell.

37. The method of claim 36, wherein the non-cardiomyocyte is obtained from bone marrow, umbilical cord blood, umbilical tissue, circulating endothelial progenitor cells, cardiac fibroblasts, adipose tissue or skin.

38. A method of treating cardiovascular disease in a subject comprising the step of delivering a composition that activates Wnt/β-catenin signaling to the cell.

39. The method of claim 38, wherein said method is further defined as:
obtaining a cell from the subject;
delivering the composition to activate Wnt/β-catenin signaling to said cell;
growing said cell to form a cell culture; and
delivering at least one cell from said cell culture to said subject.

40. The method of claim 39, wherein said delivering at least one cell from said cell culture to said subject is further defined as:
generating a tissue from said at least one cell from said cell culture; and
administering said tissue to said subject.

41. The method of claim 38, wherein the cardiovascular disease is heart failure.

42. A method of generating myocytes comprising the steps of:
obtaining non-cardiomyocyte cells;
admixing a composition that activates Wnt/β-catenin signaling; and
in vitro differentiating the cells to generate myocytes.

43. The method of claim 43, wherein obtaining said non-cardiomyocyte cells comprises performing a tissue biopsy.

44. The method of claim 43, wherein the tissue is bone marrow, umbilical cord blood, umbilical tissue, circulating endothelial progenitor cells, cardiac fibroblasts, adipose tissue or skin.

45. A method of treating a subject suffering from an infarcted myocardium comprising the step of administering to the subject an effective amount of the myocytes of claim 42, wherein the amount repairs the infarcted myocardium.

46. The method of claim 45, wherein the repairs comprise regeneration of cardiomyocytes.

47. A method of repairing an injured myocardium comprising the step of administering to a subject an effective amount of the myocytes of claim 42, wherein the amount is effective in repairing the injured myocardium.

48. The method of claim 47, wherein repairing comprises at least partially restoring structural integrity to the injured myocardium.

49. The method of claim 47, wherein repairing comprises at least partially restoring functional integrity to the injured myocardium.
